(19) United States
(12) Patent Application Publication
(10) Pub. No.: US 2025/0276013 A1
(43) Pub. Date: Sep. 4, 2025

(54) Cells Modified by Conjugated N-Terminal Glycine and Uses Thereof

(71) Applicant: Westlake Therapeutics (Hangzhou) Co. Limited, Hangzhou, Zhejiang (CN)

(72) Inventors: Xiaofei Gao, Hangzhou, Zhejiang (CN); Yanjie Huang, Hangzhou, Zhejiang (CN); Xuan Liu, Hangzhou, Zhejiang (CN); Xiaoqian Nie, Hangzhou, Zhejiang (CN)

(21) Appl. No.: 18/578,804

(22) PCT Filed: Jul. 12, 2022
(86) PCT No.: PCT/CN2022/105212

§ 371 Date: Jan. 12, 2024

(30) Foreign Application Priority Data
Jul. 13, 2021 (WO) PCT/CN2021/106079

(51) Int. Cl. A61K 35/17; A61K 35/12; A61K 35/15; A61K 35/19; A61K 45/06; A61P 35/00; C12N 5/078; C12N 9/50

(52) U.S. Cl. CPC A61K 35/17; A61K 35/15; A61K 35/19; A61K 45/06; A61P 35/00; C12N 5/0634; C12N 9/50; C12Y 304/2207; A61K 2035/124

(57) ABSTRACT

Provided is a cell having an agent linked thereto, wherein the agent is linked to at least one membrane protein of the cell via a linker comprising a N-terminal glycine through sortase recognition motif. Also provided is a method for obtaining the modified cell, as well as the use of the modified cells for delivering agents such as drugs and probes.

### TECHNICAL FIELD

The present disclosure relates generally to modified cells, and more particularly to membrane protein-modified cells and use of the same for delivering agents including drugs and probes and the like.

### BACKGROUND

Recent development in drug delivery systems for prolonging drug retention time in treating a variety of human diseases has attracted much attention. However, many of the systems still suffer from various challenges and limitations such as poor stability, unwanted toxicity, and immune responses[1]. Red blood cells (RBCs), the most common cell type in the human body, have been widely investigated as an ideal in vivo drug delivery system for over three decades due to their unique biological properties, including: (i) widespread circulation range throughout the body; (ii) good biocompatibility as a biological material with long in vivo survival time; (iii) large surface to volume ratio; and (iv) no nucleus, mitochondria, and other cellular organelles.

RBCs have been developed as drug delivery carriers by direct encapsulation, noncovalent attachment of foreign peptides, or through installation of proteins by fusion to antibodies specific for RBC surface proteins. It has been demonstrated that such modified RBCs have limitations for applications in vivo. For instance, encapsulation will disrupt cell membranes, which subsequently affects in vivo survival rates of engineered cells. In addition, the non-covalent attachment of polymeric particles to RBCs dissociates readily, and the payloads will be degraded quickly in vivo.

Bacterial sortases are transpeptidases capable of modifying proteins in a covalent and site-specific manner[2]. Wild type sortase A from Staphylococcus aureus (wt SrtA) recognizes an LPXTG motif and cleaves between threonine and glycine to form a covalent acyl-enzyme intermediate between the enzyme and the substrate protein. This intermediate is resolved by a nucleophilic attack by a peptide or protein typically having three consecutive glycine residues (3× glycines, G₃) at the N-terminus. Previous studies have genetically overexpressed a KELL membrane protein having an LPXTG motif at its C-terminus on RBCs, which can be attached to the N terminus of 3× glycines- or G_{(n≥3)}-modified proteins/peptides by using wt SrtA[3]. These RBCs carrying drugs have shown efficacy in treating diseases in animal models. However, this requires the steps of engineering hematopoietic stem or progenitor cells (HSPCs) and differentiating these cells into mature RBCs, which significantly limit the application.

Depending on applications of interest, it can be beneficial to use cells other than HSPCs to deliver therapeutic agents.

Accordingly, there is still a need in the art for an improved cell delivering system.

### SUMMARY

In a first aspect, the disclosure provides a cell having an agent linked thereto, wherein the agent is linked to at least one membrane protein of the cell via a sortase recognition motif, and the cell linked to at least one membrane protein comprises a structure of A¹-L¹-GlyₘXₙ-L²-P, in which A¹ represents the agent, L¹ represents the residual part of a sortase recognition motif after a sortase-mediated reaction, Glyₘ represents m glycines with m preferably being 1-5, Xₙ represents n spacing amino acids with n preferably being 0-10, L² is absent or represents the residual part of a first bifunctional crosslinker after crosslinking, and P represents the at least one membrane protein of the cell.

In some embodiments, the first bifunctional crosslinker is an amine-sulfhydryl type, preferably maleimido carbonic acid (C₂₋₈), e.g., 6-Maleimidohexanoic acid and 4-Maleimidobutyric acid.

In some embodiments, the first bifunctional crosslinker crosslinks said side chain amino group and at least one exposed sulfhydryl of the at least one membrane protein.

In some embodiments, the sortase recognition motif comprises, or consists essentially of, or consists of an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X is any amino acid.

In some embodiments, the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formula of CH₂OH—(CH₂)ₙ—COOH, with n being an integer from 0 to 3, and preferably n=0.

In some embodiments, the sortase recognition motif comprises, or consists essentially of, or consists of an amino acid sequence selected from the group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y, and YPXR*Y, wherein * represents the optionally substituted hydroxyl carboxylic acid, and X and Y independently represent any amino acid.

In some embodiments, the sortase recognition motif comprises, or consists essentially of, or consists of an amino acid sequence selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein M preferably is LPET*G with * being 2-hydroxyacetic acid.

In some embodiments, L¹ is selected from the group consisting of LPXT, LPXA, LPXS, LPXL, LPXV, LGXT, LAXT, LSXT, NPXT, MPXT, IPXT, SPXT, VPXT, and YPXR, with X being any amino acid.

In some embodiments, the agent A is linked to L via a second bifunctional crosslinker, which is preferably selected from a group consisting of the following types: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; more preferably the second bifunctional crosslinker is a maleimido carbonic acid (C₂₋₈), such as 6-Maleimidohexanoic acid and 4-Maleimidobutyric acid, and the agent A¹ comprises an exposed sulfydryl, preferably an exposed cysteine, more preferably a terminal cysteine, and most preferably a C-terminal cysteine.

In some embodiments, the agent comprises a binding agent, a therapeutic agent, or a detection agent, including for example a protein, a peptide such as an extracellular domain of oligomeric ACE2, an antibody such as anti-PD1 antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme) such as Aspergillus flavus uricase, a hormone, a cytokine, a growth factor, an antimicrobial agent, a probe, a ligand, a receptor, an immunotolerance-inducing peptide, a targeting moiety, a prodrug, or any combination thereof.

In some embodiments, the cell comprises a structure of A¹-LPET-GlyₘXₙ-L²-P, preferably, the A selected from PAL (phenylalanine ammonia-lyase), HPV (such as HPV16-MHC1), UOX, or PD1 mAb, more preferably, the GlyₘXₙ-L² is GAASK-mal.

In some embodiments, the sortase is a Sortase A (SrtA) such as a Staphylococcus aureus transpeptidase A, e.g., Staphylococcus aureus transpeptidase A variant (mgSrtA).

In some embodiments, the cell is selected from the group consisting of red blood cells, T cells, B cells, monocytes, NK cells, and megakaryocytes.

In some embodiments, wherein the cell is red blood cells with a structure selected from PAL-LPET-GAASK-mal-P, HPV-LPET-GAASK-mal-P, HPV16-MHC1-LPET-GAASK-mal-P, UOX-LPET-GAASK-mal-P, or PD1 mAb-1-LPET-GAASK-mal-P.

In a second aspect, the disclosure provides a method for modifying a cell, comprising:
(i) providing a peptide having the formula GlyₘXₙ-L², in which Glyₘ represents m glycines with m preferably being 1-5, and Xₙ represents n spacing amino acids with n preferably being 0-10, and L^{2′} represents the residual part of a first bifunctional crosslinker after linking to GlyₘXₙ;
(ii) treating a cell with the GlyₘXₙ-L² peptide under a condition to link the GlyₘXₙ-L² peptide to at least one membrane protein of the cell; and
(iii) contacting the treated cell with a sortase substrate that comprises a sortase recognition motif and an agent, in the presence of a sortase under one or more conditions suitable for the sortase to conjugate the sortase substrate to the Glyₘ by a sortase-mediated reaction,
   thereby a modified cell with a structure of A¹-L¹-GlyₘXₙ-L²-P is obtained, wherein A represents the agent, L¹ represents the residual part of a sortase recognition motif after a sortase-mediated reaction, and P represents the at least one membrane protein of the cell.

In some embodiments, before the treating step, the method further comprises a step of pretreating the cell with a reducing agent to form an exposed sulfhydryl.

In some embodiments, the first bifunctional crosslinker is an amine-sulfhydryl type, preferably maleimido carbonic acid (C₂₋₈), e.g., 6-Maleimidohexanoic acid and 4-Maleimidobutyric acid.

In some embodiments, Xₙ comprises at least one amino acid having a side chain amino group such as lysine, and preferably the C-terminal amino acid of Xₙ is an amino acid having a side chain amino group.

In some embodiments, the first crosslinker crosslinks said side chain amino group and at least one exposed sulfhydryl of the at least one membrane protein.

In some embodiments, the sortase recognition motif comprises, or consists essentially of, or consists of an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X is any amino acid.

In some embodiments, the sortase recognition motif comprises an unnatural amino acid located at position 5 from the direction of N-terminal to C-terminal of the sortase recognition motif, wherein the unnatural amino acid is an optionally substituted hydroxyl carboxylic acid having a formula of CH₂OH—(CH₂)ₙ—COOH, with n being an integer from 0 to 3, and preferably n=0.

In some embodiments, the sortase recognition motif comprises, or consists essentially of, or consists of an amino acid sequence selected from the group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y, and YPXR*Y, wherein * represents the optionally substituted hydroxyl carboxylic acid; and X and Y independently represent any amino acid.

In some embodiments, the sortase recognition motif comprises, or consists essentially of, or consists of an amino acid sequence selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein M preferably is LPET*G with * being 2-hydroxyacetic acid.

In some embodiments, the agent A is linked to L¹ via a second bifunctional crosslinker, which is preferably selected from the group consisting of the following types: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; more preferably the second bifunctional crosslinker is a maleimido carbonic acid (C₂₋₈), such as 6-Maleimidohexanoic acid and 4-Maleimidobutyric acid, and the agent A¹ comprises an exposed sulfydryl, preferably an exposed cysteine, more preferably a terminal cysteine, and most preferably a C-terminal cysteine.

In some embodiments, the agent comprises a binding agent, a therapeutic agent, or a detection agent, including for example a protein, a peptide such as an extracellular domain of oligomeric ACE2, an antibody such as anti-PD1 antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme) such as Aspergillus flavus uricase, a hormone, a cytokine, a growth factor, an antimicrobial agent, a probe, a ligand, a receptor, an immunotolerance-inducing peptide, a targeting moiety, a prodrug, or any combination thereof.

In some embodiments, the sortase is a Sortase A (SrtA) such as a Staphylococcus aureus transpeptidase A, e.g., Staphylococcus aureus transpeptidase A variant (mgSrtA).

In some embodiments, the modified cell comprises a structure of A¹-LPET-GlyₘXₙ-L²-P, preferably, the A selected from PAL (phenylalanine ammonia-lyase), HPV (such as HPV16-MHC1), UOX, or PD1 mAb, more preferably, the GlyₘXₙ-L² is GAASK-mal.

In some embodiments, the cell is selected from the group consisting of red blood cells, T cells, B cells, monocytes, NK cells, and megakaryocytes.

In some embodiments, wherein the modified cell is red blood cells with a structure selected from PAL-LPET-GAASK-mal-P, HPV-LPET-GAASK-mal-P, HPV16-MHC1-LPET-GAASK-mal-P, UOX-LPET-GAASK-mal-P, or PD1 mAb-1-LPET-GAASK-mal-P.

In a third aspect, the disclosure provides a cell obtained by the method of the second aspect.

In a fourth aspect, the disclosure provides a composition comprising the cell of the first and/or third aspect and optionally a physiologically acceptable carrier.

In a fifth aspect, the disclosure provides a method for diagnosing, treating, or preventing a disorder, condition, or disease in a subject in need thereof, comprising administering the cell of the first and/or third aspect or the composition of the fourth aspect to the subject.

In some embodiments, the disorder, condition, or disease is selected from the group consisting of tumors or cancers such as cervical cancer, metabolic diseases such as lysosomal storage disorders (LSDs), bacterial infections, virus infections such as coronavirus infection for example SARS-COV or SARS-COV-2 infection, autoimmune diseases, and inflammatory diseases.

In a sixth aspect, the disclosure provides a method of delivering an agent to a subject in need thereof, comprising administering the cell of the first and/or third aspect or the composition of the fourth aspect to the subject.

In a seventh aspect, the disclosure provides a method of increasing the circulation time or plasma half-life of an agent in a subject, comprising attaching the agent to a cell according to the method of the second aspect.

In an eighth aspect, the invention provides a use of the cell of the first and/or third aspect or the composition of the fourth aspect in the manufacture of a medicament for diagnosing, treating, or preventing a disorder, condition, or disease, or a diagnostic agent for diagnosing a disorder, condition, or disease or for delivering an agent.

In some embodiments, the disorder, condition, or disease is selected from the group consisting of tumors or cancers such as cervical cancer, metabolic diseases such as lysosomal storage disorders (LSDs), bacterial infections, virus infections such as coronavirus infection for example SARS-COV or SARS-COV-2 infection, autoimmune diseases, and inflammatory diseases.

In some embodiments, the medicament is a vaccine.

In a ninth aspect, the cell of the first and/or third aspect or the composition of the fourth aspect is provided for use in diagnosing, treating, or preventing a disorder, condition, or disease in a subject in need thereof.

In some embodiments, the disorder, condition, or disease is selected from the group consisting of tumors or cancers such as cervical cancer, metabolic diseases such as lysosomal storage disorders (LSDs), bacterial infections, virus infections such as coronavirus infection for example SARS-COV or SARS-COV-2 infection, autoimmune diseases, and inflammatory diseases.

### BRIEF DESCRIPTION OF THE DRAWINGS

In the drawings, embodiments of the present disclosure are illustrated by way of example. It is to be expressly understood that the description and drawings are only for the purpose of illustration and as an aid to understanding the present disclosure, and are not intended as a definition of the limits of the invention.

FIG. 1 illustrates an exemplary process for labeling red blood cells with a peptide in vitro according to one embodiment of the present disclosure.

FIG. 2 shows the structural formula of GAASK-mal (also named as GAASK-6-mal).

FIG. 3 shows the efficient labeling of eGFP-LPETGAASK-mal on the surface of natural RBCs that was detected by flow cytometry. Control group: Unlabled RBCs; Treatment Group: RBCs labeled with eGFP-LEPTG, and RBCs labeled with eGFP-LPETGAASK-mal. Histograms show eGPF signals on the RBCs' surface after their incubation with corresponding molecule, respectively.

FIG. 4 shows the percentage of RBCs with eGFP signals in vivo. 10⁹ mouse RBCs were labeled with eGFP-LPETGAASK-mal. The labeled RBCs were stained with cell trace Far Red dye and injected intravenously into the mice. Mice were bled at 21 days post transfusion. Blood samples were analyzed by flow cytometry. Far Red positive cells were selected for analyzing the percentage of RBCs with eGFP signals.

FIGS. 5A-5B show the percentage of eGFP positive cells in the circulation and the label stability of these RBCs in different days. FIG. 5A Recipient mice were bled at indicated days post transfusion. Far Red positive cells indicate the percentage of transfused RBCs in the circulation. FIG. 5B Far Red positive RBCs from the blood samples of the above experiments were analyzed for measuring the label stability of these eGFP positive RBCs.

FIG. 6 shows the efficient labeling of PAL-LPETGAASK-mal on the surface of natural RBCs that was detected by flow cytometry. Control group: Unlabeled RBCs; Treatment Group: RBCs labeled with PAL-LEPTG, and RBCs labeled with PAL-LPETGAASK-mal. Histograms show PAL signals on the RBCs' surface after their incubation with corresponding molecule, respectively.

FIGS. 7A-7B show the percentage of PAL positive cells in the circulation and the label stability of these RBCs in different days. FIG. 7A Recipient mice were bled at indicated days post transfusion. Survival percentage of injected RBCs was indicated by Far red labeled positive cells. FIG. 7B Far Red positive RBCs from the blood samples of the above experiments were analyzed for measuring the label stability of these PAL positive RBCs.

FIG. 8 shows the efficient labeling of HPV16-hMHC1-LPETGAASK-mal on the surface of natural RBCs that was detected by flow cytometry. Control group: Unlabeled RBCs; Treatment Group: RBCs labeled with HPV16-hMHC1-LPETG, and RBCs labeled with HPV16-hMHC1-LPETGAASK-mal. FIG. 8A is Histogram showing HPV16-hMHC1 signals on the RBCs' surface after their incubation with corresponding molecule, respectively; FIG. 8B, Recipient mice were bled at indicated days post transfusion. Far Red positive cells indicate the percentage of transfused RBCs in the circulation. FIG. 8C, Far Red positive RBCs from the blood samples of the above experiments were analyzed for measuring the label stability of these MHC1 positive RBCs.

FIG. 9 shows the efficient labeling of eGFP-LPETGAASK-mal on the surface of other mammalian cells. After T cell, Monocytes, NK cells, B cells, and Megakaryocytes being labeled with eGFP-LPETGAASK-mal, respectively, the labeling efficacy was detected by flow cytometry. T cells are Anti-CD3 positive cells; monocytes are Anti-CD14 positive cells; NK cells are Anti-CD16 positive cells; B cells are Anti-CD19 positive cells; megakaryocytes are Anti-CD41 positive indicates cells.

FIG. 10 shows that SARS-CoV-2 enters host cells through binding with ACE2 by its S protein.

FIG. 11 shows red blood cell (RBC) with trimeric ACE2 engineered on the surface.

FIG. 12 shows the chemical structure of the irreversible linker 6-Mal-LPET*G (6-Maleimidohexanoic acid-Leu-Pro-Glu-Thr-2-hydroxyacetic acid-Gly); 6-Mal represents 6-Maleimidohexanoic acid.

FIG. 13 shows a reaction scheme for the conjugation of the irreversible linker 6-Mal-LPET*G to a modified protein. The two reaction substrates are mixed and reacted in a ratio of 1:4=eGFP-cys:6-Mal-LPET*G to obtain the final reaction product.

FIG. 14 shows the chemical structure of the irreversible linkers 6-Mal-K(6-Mal)-GGG-K(6-Mal)-GGGSAA-LPET*G and 6-Mal-K(6-Mal)-GGGGGGSAA-LPET*G (top) and a schematic diagram of a protein conjugated by a double fork and triple fork (bottom).

FIG. 15 shows the blockade efficacy of anti PD1 mAb-1-RBCs and anti PD1 mAb-2-RBCs in vitro.

FIG. 16 shows the anti-tumor efficacy of anti PD1 mAb-2-RBCs in vivo. (A) The tumor volume of mice during therapy; (B) the body weight of mice during therapy; (C) quantitative analysis of tumor weight on day 22; and (D) photos of the excised tumors on day 22

FIG. 17 shows the pharmacokinetics study of anti PD1 mAb-1-RBCs in vivo.

FIG. 18 shows the characterization of UOX-RBCs during storage. The left figure shows hemolysis ration of individual UOX-RBCs, and the right figures shows deformability of individual UOX-RBCs.

FIG. 19 shows the enzymatic activity of UOX conjugated on RBCs in vitro, in which the data shown enzymatic activity of UOX after minus corresponding enzymatic activity of control RBCs.

FIG. 20 shows the efficacy of UOX-RBCs in vivo in a mouse model of gout.

FIG. 21 shows pharmacokinetics study of UOX-RBCs in vivo.

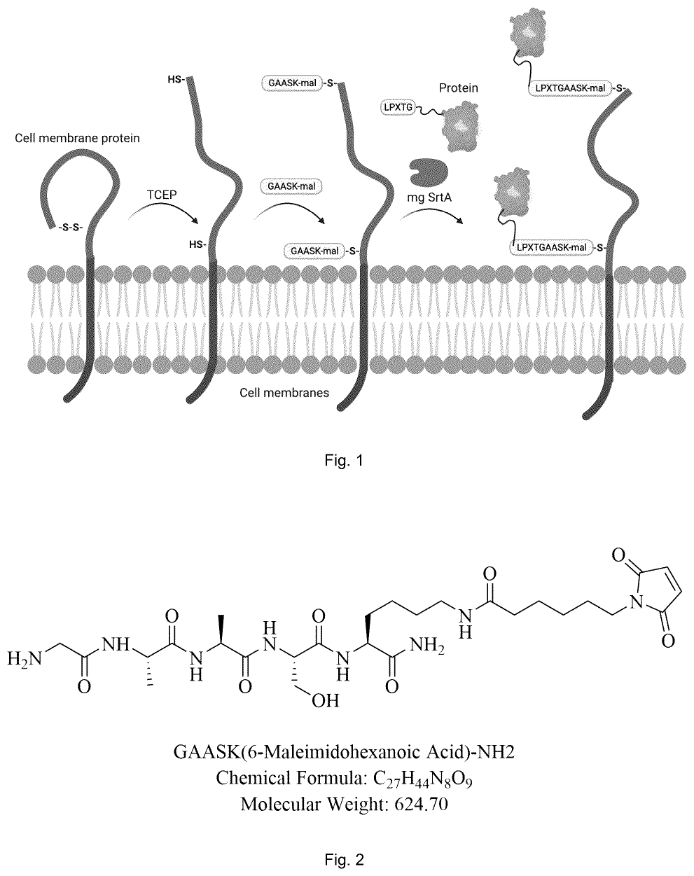

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, unless otherwise specified, the scientific and technical terms used herein have the meanings as generally understood by a person skilled in the art. Although any methods and materials similar or equivalent to those described herein find use in the practice of the present invention, the preferred methods and materials are described herein. Accordingly, the terms defined herein are more fully described by reference to the Specification as a whole.

As used herein, the singular terms “a,” “an,” and “the” include the plural reference unless the context clearly indicates otherwise.

Unless otherwise indicated, nucleic acids are written left to right in 5′ to 3′ orientation; and amino acid sequences are written left to right in amino to carboxy orientation, respectively.

It is to be understood that this invention is not limited to the particular methodology, protocols, and reagents described, as these may vary, depending upon the context in which they are used by those of skills in the art.

As used herein, the term “consisting essentially of” in the context of an amino acid sequence is meant the recited amino acid sequence together with additional one, two, three, four, or five amino acids at the N- or C-terminus.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

As used herein, the terms “patient,” “individual,” and “subject” are used in the context of any mammalian recipient of a treatment or composition disclosed herein. Accordingly, the methods and composition disclosed herein may have medical and/or veterinary applications. In a preferred form, the mammal is a human.

As used herein, the term “sequence identity” is meant to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, California, USA).

As used herein, the term “conjugated” or “conjugation” refers to an association of two molecules, for example, two proteins or a protein and a small molecule or other entity, with one another in a way that they are linked by a direct or indirect covalent or non-covalent interaction.

The inventors have herein developed a new strategy to modify cells, e.g., blood cells, especially natural RBCs, with an agent, e.g., peptides and/or small molecules through a sortase-mediated reaction. The technology allows for producing cell products by directly modifying natural cells such as RBCs instead of HSPCs, which are limited by their resources, at a very high labelling efficiency. Also, the modified cells preserve their original biological properties well and remain as stable as their native state. In some embodiments, the labelled red blood cells have the same lifespan as native RBCs and sustained signals in circulation for up to 28 days or longer.

### Cells

In one aspect, the present disclosure provides a cell having an agent linked thereto, wherein the agent is linked to at least one membrane protein of the cell via a sortase recognition motif. To increase the labelling efficiency of a sortase-mediated reaction, a linker comprising an N-terminal glycine is conjugated, via a bifunctional crosslinker such as an amine-sulfhydryl type bifunctional crosslinker, to at least one membrane protein of a cell, preferably to at least one exposed sulfhydryl of the at least one membrane protein.

Contemplated in the present disclosure are various living animal cells, such as mammalian cells, e.g., various blood cells, including red blood cells, T cells, B cells, monocytes, NK cells and megakaryocytes. In some embodiments the animal cells are mammalian cells, e.g., human cells. In some embodiments the cells are immune system cells, e.g., lymphocytes (e.g., T cells or NK cells) or dendritic cells. In some embodiments, the cells are a cytotoxic cell. In some embodiments, the cells are non-immortalized cells. In some embodiments, the cells are primary cells. In some embodiments, the cells are natural cells. In some embodiments, the cells are not genetically engineered to express a polypeptide comprising a sortase recognition sequence, a sequence comprising one or more glycines or alanines at its N terminus or C terminus, or both.

In some embodiments, the cell is a mature red blood cell (RBC). In certain embodiments, the RBC is a human RBC, such as a human natural RBC. In some embodiments, the RBC is a red blood cell that has not been genetically engineered to express a protein comprising a sortase recognition motif or a nucleophilic acceptor sequence. In some embodiments the RBC has not been genetically engineered.

The terms “non-engineered, “non-genetically modified,” and “non-recombinant” as used herein are interchangeable and refer to not being genetically engineered, absence of genetic modification, etc. Non-engineered membrane proteins encompass endogenous proteins. In certain embodiments, a non-genetically engineered red blood cell does not contain a non-endogenous nucleic acid, e.g., DNA or RNA that originates from a vector, from a different species, or that comprises an artificial sequence, e.g., DNA or RNA that was introduced artificially. In certain embodiments, a non-engineered cell has not been intentionally contacted with a nucleic acid that is capable of causing a heritable genetic alteration under conditions suitable for uptake of the nucleic acid by the cell.

In some embodiments, the cells have not been genetically engineered for sortagging. A cell is considered “not genetically engineered for sortagging” if the cell has not been genetically engineered to express a protein comprising a sortase recognition motif or a nucleophilic acceptor sequence in a sortase-catalyzed reaction.

In some aspects, the present disclosure provides a cell having an agent linked thereto, wherein the agent is linked to at least one membrane protein of the cell via a sortase recognition motif, and the agent linked to the at least one membrane protein comprises a structure of A¹-L¹-GlyₘXₙ-L²-P, in which A represents the agent, L¹ represents the residual part of a sortase recognition motif after a sortase-mediated reaction, Glyₘ represents m glycines with m preferably being 1-5, Xₙ represents n spacing amino acids with n preferably being 0-10, L² is absent or represents the residual part of a first bifunctional crosslinker after crosslinking, and P represents the at least one membrane protein of the cell.

In some embodiments, the present disclosure provides a cell having an agent linked thereto as described herein. In some embodiments, a composition comprising a plurality of such cells is provided. In some embodiments, at least a selected percentage of the cells in the composition are modified, e.g., by having an agent linked to at least one membrane protein of the cell. For example, in some embodiments at least 5%, 10%, 15%, 20%, 25%, 30%, 40%, 50%, 60%, 70%, 80%, 90%, 95%, 96%, 97%, 98%, 99%, or more of the cells have an agent linked thereto. In some embodiments, the linked agent may be one or more of the agents described herein.

In some embodiments, the present disclosure provides a cell that comprises an agent linked to the membrane protein of the cell by using a sortase recognition motif and a linker comprising a terminal glycine such as an N-terminal glycine (e.g., a linker having a structure of GlyₘXₙ, wherein Glyₘ represents m glycines with m preferably being 1-5, and Xₙ represents n spacing amino acids with n preferably being 0-10). In some embodiments, the agents linked to cells may be the same species or different species.

In some embodiments, the agent is linked via a sortase recognition motif to a linker comprising a terminal glycine such as an N-terminal glycine (e.g. a linker having a structure of GlyₘXₙ, wherein Glyₘ represents m glycines with m preferably being 1-5, and Xₙ represents n spacing amino acids with n preferably being 0-10) that is crosslinked to at least one membrane protein of a cell. In some embodiments, the sortase recognition motif may be selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X is any amino acid. In some embodiments, the sortase recognition motif may comprise an unnatural amino acid located at position 5 from the direction of N-terminal to C-terminal of the sortase recognition motif, wherein the unnatural amino acid is an optionally substituted hydroxyl carboxylic acid having a formulae of CH₂OH—(CH₂)ₙ—COOH, with n being an integer from 0 to 3, and preferably n=0. In some embodiments, the sortase recognition motif comprising an unnatural amino acid may be selected from the group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y, and YPXR*Y, wherein * represents the optionally substituted hydroxyl carboxylic acid and X and Y independently represent any amino acid. In some embodiments, the sortase recognition motif comprising an unnatural amino acid may be selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein Motif (M) preferably is LPET*G with * preferably being 2-hydroxyacetic acid.

It can be understood that after the agent is linked to the membrane protein, the last one or two residues from 5^{th} position (from the direction of N-terminal to C-terminal) of the sortase recognition motif is replaced by the amino acid on which the linkage occurs, as described elsewhere herein. For example, the agent linked to the membrane protein may comprise a structure of A¹-L¹-GlyₘXₙ-L²-P, in which L¹ represents the residual part of a sortase recognition motif after a sortase-mediated reaction, and may be selected from the group consisting of LPXT, LPXA, LPXS, LPXL, LPXV, LGXT, LAXT, LSXT, NPXT, MPXT, IPXT, SPXT, VPXT, and YPXR.

In some embodiments, genetically engineered cells are modified by using sortase to attach or conjugate or link a sortase substrate to the membrane protein of the cells. The cell (e.g., RBCs), for example, have been genetically engineered to express any of a wide variety of products, e.g., polypeptides or noncoding RNAs, may be genetically engineered to have a deletion of at least a portion of one or more genes, and/or may be genetically engineered to have one or more precise alterations in the sequence of one or more endogenous genes. In certain embodiments, such a genetically engineered cell is sortagged with any of the various agents described herein according to the method described herein.

In some embodiments, the present disclosure contemplates using autologous cells e.g., red blood cells, that are isolated from an individual to whom such isolated cells, after being modified in vitro, are to be administered. In some embodiments, the present disclosure contemplates using immuno-compatible red blood cells that are of the same blood group as an individual to whom such cells are to be administered (e.g., at least with respect to the ABO blood type system and, in some embodiments, with respect to the D blood group system) or may be of a compatible blood group.

### A Linker Comprising a Terminal Glycine

In some embodiments, a linker comprising a terminal glycine such as an N-terminal glycine (e.g., a linker having a structure of GlyₘXₙ, wherein Glyₘ represents m glycines with m preferably being 1-5, and Xₙ represents n spacing amino acids with n preferably being 0-10) is linked via a first bifunctional crosslinker to at least one membrane protein of the cell.

As used herein, Glyₘ refers to m glycines with m preferably being 1-5, such as one, two, three, four, or five glycines. As used herein, Xₙ represents n optional spacing amino acids which can be any amino acids. In some embodiments, n can be 0-10 or more, such as 0-5, 1-4, or 0, 1, 2, 3, 4, 5, 6, 7, 8, 9, or 10. In some embodiments, the spacing amino acids may be any natural or unnatural amino acids, such as Gly, Ala, Ser, Lys, Asn, Thr, Glu, or Gln. In some embodiments, Xₙ may be AAS or AASK.

As used herein, the term “a bifunctional crosslinker” refers to a reagent that is designed to link two reactive groups. If a bifunctional crosslinker is designed so that its two reactive groups are identical, it is referred to as a homobifunctional crosslinker; if its two reactive groups are different, it is a heterobifunctional crosslinker. If one or both reactive groups of the crosslinker become so only as the result of a photochemical reaction caused by exposing the crosslinker to light of an appropriate wavelength, then the crosslinker is photoactivatable, photoreactive, photosensitive, or light-activated. The present disclosure contemplates using various bifunctional crosslinkers that are capable of crosslinking the linker comprising a terminal glycine to the at least one membrane protein of the cell.

In some embodiments, the bifunctional crosslinker may include but are not limited to: (1) zero-length type (e.g., EDC; EDC plus sulfo NHS; CMC; DCC; DIC; N,N′-carbonyldiimidazole; Woodward's reagent K); (2) amine-sulfhydryl type such as an NHS ester-maleimide heterobifunctional crosslinker; Maleimido carbonic acid (C₂₋₈) (e.g., 6-Maleimidohexanoic acid and 4-Maleimidobutyric acid); EMCS; SPDP, LC-SPDP, sulfo-LC-SPDP; SMPT and sulfo-LC-SMPT; SMCC, LC-SMCC and sulfo-SMCC; MBS and sulfo-MBS; SIAB and sulfo-SIAB; SMPB and sulfo-SMPB; GMBS and sulfo-GMBS; SIAX and SIAXX; SIAC and SIACX; NPIA; (3) homobifunctional NHS esters type (e.g., DSP; DTSSP; DSS; DST and Sulfo-DST; BSOCOES and Sulfo-BSOCOES; EGS and Sulfo-EGS); (4) homobifunctional imidoesters type (e.g., DMA; DMP; DMS; DTBP); (5) carbonyl-sulfydryl type (e.g., KMUH; EMCH; MPBH; M2C2H; PDPH); (6) sulfhydryl reactive type (e.g., DPDPB; BMH; HBVS); (7) sulfhydryl-hydroxy type (e.g., PMPI); or the like.

In some embodiments, the bifunctional crosslinker is an amine-sulfhydryl type, preferably maleimido carbonic acid (C₂₋₈), e.g., 6-Maleimidohexanoic acid and 4-Maleimidobutyric acid. In some further embodiments, the linker comprising a terminal glycine comprises at least one amino acid having a side chain amino group such as lysine, and preferably the C-terminal amino acid of Xₙ is an amino acid having a side chain amino group, which enables the crosslinking reaction to occur between the side chain amino group and at least one exposed sulfhydryl of the at least one membrane protein.

### Sortase

Enzymes identified as “sortases” have been isolated from a variety of Gram-positive bacteria. Sortases, sortase-mediated transacylation reactions, and their use in protein engineering are well known to those of ordinary skilled in the art (see, e.g., PCT/US2010/000274 (WO/2010/087994), and PCT/US2011/033303 (WO/2011/133704)). Sortases have been classified into 4 classes, designated A, B, C, and D, based on sequence alignment and phylogenetic analysis of 61 sortases from Gram-positive bacterial genomes (Dramsi S, Trieu-Cuot P, Bierne H, Sorting sortases: a nomenclature proposal for the various sortases of Gram-positive bacteria. Res Microbiol. 156(3):289-97, 2005). Those skilled in the art can readily assign a sortase to the correct class based on its sequence and/or other characteristics such as those described in Drami, et al., supra. The term “sortase A” as used herein refers to a class A sortase, usually named SrtA in any particular bacterial species, e.g., SrtA from S. aureus or S. pyogenes.

The term “sortase,” also known as transamidases, refers to an enzyme that has transamidase activity. Sortases recognize substrates comprising a sortase recognition motif, e.g., the amino acid sequence LPXTG. A molecule recognized by a sortase (i.e., comprising a sortase recognition motif) is sometimes termed a “sortase substrate” herein. Sortases tolerate a wide variety of moieties in proximity to the cleavage site, thus allowing for the versatile conjugation of diverse entities so long as the substrate contains a suitably exposed sortase recognition motif and a suitable nucleophile is available. The terms “sortase-mediated transacylation reaction,” “sortase-catalyzed transacylation reaction,” “sortase-mediated reaction,” “sortase-catalyzed reaction,” “sortase reaction,” “sortase-mediated transpeptide reaction,” and like terms are used interchangeably herein to refer to such a reaction. The terms “sortase recognition motif,” “sortase recognition sequence,” and “transamidase recognition sequence,” with respect to sequences recognized by a transamidase or sortase, are used interchangeably herein. The term “nucleophilic acceptor sequence” refers to an amino acid sequence capable of serving as a nucleophile in a sortase-catalyzed reaction, e.g., a sequence comprising an N-terminal glycine (e.g., 1, 2, 3, 4, or 5 N-terminal glycines).

The present disclosure encompasses embodiments relating to any of the sortase classes known in the art (e.g., a sortase A, B, C, or D from any bacterial species or strain). In some embodiments, sortase A is used, such as SrtA from S. aureus. In some embodiments two or more sortases may be used. In some embodiments the sortases may utilize different sortase recognition sequences and/or different nucleophilic acceptor sequences.

In some embodiments, the sortase is a sortase A (SrtA). SrtA recognizes the motif LPXTG, with common recognition motifs being, e.g., LPKTG, LPATG, LPNTG. In some embodiments LPETG is used. However, motifs falling outside this consensus may also be recognized. For example, in some embodiments the motif comprises an A, S, L, or V rather than a T at position 4, e.g., LPXAG, LPXSG, LPXLG, or LPXVG, e.g., LPNAG or LPESG, LPELG, or LPEVG. In some embodiments the motif comprises an A rather than a G at position 5, e.g., LPXTA, e.g., LPNTA. In some embodiments the motif comprises a G or A rather than P at position 2, e.g., LGXTG or LAXTG, e.g., LGATG or LAETG. In some embodiments the motif comprises an I or M rather than L at position 1, e.g., MPXTG or IPXTG, e.g., MPKTG, IPKTG, IPNTG or IPETG. Diverse recognition motifs of sortase A are described in Pishesha et al. 2018.

In some embodiments, the sortase recognition sequence is LPXTG, wherein X is a standard or non-standard amino acid. In some embodiments, X is selected from D, E, A, N, Q, K, or R. In some embodiments, the recognition sequence is selected from LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X may be any amino acids, such as those selected from D, E, A, N, Q, K, or R in certain embodiments.

In some embodiments, the sortase may recognize a motif comprising an unnatural amino acid, preferably located at position 5 from the direction of N-terminal to C-terminal of the sortase recognition motif. In some embodiments, the unnatural amino acid is a substituted or unsubstituted hydroxyl carboxylic acid having a formulae of CH₂OH—(CH₂)ₙ—COOH, with n being an integer from 0 to 5, e.g., 0, 1, 2, 3, 4, and 5, and preferably n=0. In some embodiments, the unnatural amino acid is a substituted hydroxyl carboxylic acid and in some further embodiments, the hydroxyl carboxylic acid is substituted by one or more substituents selected from halo, C₁₋₆ alkyl, C₁₋₆ haloalkyl, hydroxyl, C₁₋₆ alkoxy, and C₁₋₆ haloalkoxy. The term “halo” or “halogen” means fluoro, chloro, bromo, or iodo, and preferred are fluoro and chloro. The term “alkyl” by itself or as part of another substituent refers to a hydrocarbyl radical of Formula CₙH₂ₙ₊₁ wherein n is a number greater than or equal to 1. In some embodiments, alkyl groups useful in the present disclosure comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be further substituted as indicated herein. C_{x-y} alkyl refers to alkyl groups which comprise from x to y carbon atoms. Suitable alkyl groups include methyl, ethyl, n-propyl, i-propyl, n-butyl, i-butyl, s-butyl, tert-butyl, pentyl and its isomers (e.g. n-pentyl, iso-pentyl), and hexyl and its isomers (e.g. n-hexyl, iso-hexyl). Preferred alkyl groups include methyl, ethyl, n-propyl, i-propyl, n-butyl, i-butyl, s-butyl, and tert-butyl. The term “haloalkyl” alone or in combination, refers to an alkyl radical having the meaning as defined above, wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl, and the like.

In some embodiments, the sortase recognition motif comprising an unnatural amino acid may be selected from the group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y, and YPXR*Y, wherein * represents the optionally substituted hydroxyl carboxylic acid, and X and Y independently represent any amino acid. In some embodiments, the sortase recognition motif comprising an unnatural amino acid may be selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein M preferably is LPET*G with * preferably being 2-hydroxyacetic acid.

In some embodiments, the present disclosure contemplates using a variant of a naturally occurring sortase. In some embodiments, the variant is capable of mediating a glycine₍ₙ₎ conjugation, with n preferably being 1 or 2. Such variants may be produced through processes such as directed evolution, site-specific modification, etc. Considerable structural information regarding sortase enzymes, e.g., sortase A enzymes, is available, including NMR or crystal structures of SrtA alone or bound to a sortase recognition sequence (see, e.g., Zong Y, et al. J. Biol Chem. 2004, 279, 31383-31389). The active site and substrate binding pocket of S. aureus SrtA have been identified. One of ordinary skilled in the art can generate functional variants by, for example, avoiding deletions or substitutions that would disrupt or substantially alter the active site or substrate binding pocket of a sortase. In some embodiments, directed evolution on SrtA can be performed by utilizing the FRET (Fluorescence Resonance Energy Transfer)-based selection assay described in Chen, et al. Sci. Rep. 2016, 6 (1), 31899. In some embodiments, a functional variant of S. aureus SrtA may be those described in CN106191015A and CN109797194A. In some embodiments, the S. aureus SrtA variant can be a truncated variant with, e.g., 25-60 (e.g., 30, 35, 40, 45, 50, 55, 59, or 60) amino acids being removed from N-terminus (as compared to the wild type S. aureus SrtA).

In some embodiments, a functional variant of S. aureus SrtA useful in the present disclosure may be a S. aureus SrtA variant comprising one or more mutations on amino acid positions of D124, Y187, E189, and F200 of D124G, such as Y187L, E189R, and F200L and optionally further comprising one or more mutations of P94S/R, D160N, D165A, K190E, and K196T. In certain embodiments, the S. aureus SrtA variant may comprise D124G; D124G and F200L; P94S/R, D124G, D160N, D165A, K190E, and K196T; P94S/R, D160N, D165A, Y187L, E189R, K190E, and K196T; P94S/R, D124G, D160N, D165A, Y187L, E189R, K190E, and K196T; D124G, Y187L, E189R, and F200L; or P94S/R, D124G, D160N, D165A, Y187L, E189R, K190E, K196T, and F200L. In some embodiments, the S. aureus SrtA variants have 25 to 60 (e.g., 25, 30, 35, 40, 45, 50, 55, 59, or 60) amino acids being removed from the N-terminus. In some embodiments, the mutated amino acid positions above are numbered according to the numbering of a wild type S. aureus SrtA, e.g., as shown in SEQ ID NO: 1. In some embodiments, the full-length nucleotide sequence of the wild type S. aureus SrtA is shown as in e.g., SEQ ID NO: 2.

| (full length, GenBank Accession No .: CAA3829591.1) | |
|---|---|
| SEQ ID NO: 1 | |
| 1 | MKKWTNRLMT IAGVVLILVA AYLFSKPHID NYLHDKDKDE KIEQYDKNVK |
| 51 | EQASKDKKQQ AKPQIPKDKS KVAGYIEIPD ADIKEPVYPG PATPEQLNRG |
| 101 | VSFAEENESL DDQNISIAGH TFIDRPNYQF TNLKAAKKGS MVYFKVGNET |
| 151 | RKYKMTSIRD VKPTDVGVLD EQKGKDKQLT LITCDDYNEK TGVWEKRKIF |
| 201 | VATEVK |

(full length, wild type)
SEQ ID NO: 2
ATGAAAAAATGGACAAATCGATTAATGACAATCGCTGGTGTGGTACTTATCCTAG
TGGCAGCATATTTGTTTGCTAAACCACATATCGATAATTATCTTCACGATAAAGA
TAAAGATGAAAAGATTGAACAATATGATAAAAATGTAAAAGAACAGGCGAGTA
AAGATAAAAAGCAGCAAGCTAAACCTCAAATTCCGAAAGATAAATCGAAAGTGG
CAGGCTATATTGAAATTCCAGATGCTGATATTAAAGAACCAGTATATCCAGGACC
AGCAACACCTGAACAATTAAATAGAGGTGTAAGCTTTGCAGAAGAAAATGAATC
ACTAGATGATCAAAATATTTCAATTGCAGGACACACTTTCATTGACCGTCCGAAC
TATCAATTTACAAATCTTAAAGCAGCCAAAAAAGGTAGTATGGTGTACTTTAAAG
TTGGTAATGAAACACGTAAGTATAAAATGACAAGTATAAGAGATGTTAAGCCTA
CAGATGTAGGAGTTCTAGATGAACAAAAAGGTAAAGATAAACAATTAACATTAA
TTACTTGTGATGATTACAATGAAAAGACAGGCGTTTGGGAAAAACGTAAAATCTT
TGTAGCTACAGAAGTCAAA

In some embodiments, as compared to a wild type S. aureus SrtA, the S. aureus SrtA variant may comprise one or more mutations at one or more of the positions corresponding to 94, 105, 108, 124, 160, 165, 187, 189, 190, 196, and 200 of SEQ ID NO: 1. In some embodiments, as compared to a wild type S. aureus SrtA, the S. aureus SrtA variant may comprise one or more mutations corresponding to P94S/R, E105K, E108A, D124G, D160N, D165A, Y187L, E189R, K190E, K196T, and F200L. In some embodiments, as compared to a wild type S. aureus SrtA, the S. aureus SrtA variant may comprise one or more mutations corresponding to D124G, Y187L, E189R, and F200L, and optionally further comprises one or more mutations corresponding to P94S/R, D160N, D165A, K190E, and K196T and optionally further one or more mutations corresponding to E105K and E108A. In certain embodiments, as compared to a wild type S. aureus SrtA, the S. aureus SrtA variant may comprise mutations corresponding to D124G; D124G and F200L; P94S/R, D124G, D160N, D165A, K190E, and K196T; P94S/R, D160N, D165A, Y187L, E189R, K190E, and K196T; P94S/R, D124G, D160N, D165A, Y187L, E189R, K190E, and K196T; D124G, Y187L, E189R, and F200L; or P94S/R, D124G, D160N, D165A, Y187L, E189R, K190E, K196T, and F200L. In some embodiments, the S. aureus SrtA variant may comprise one or more mutations of P94S/R, E105K, E108A, D124G, D160N, D165A, Y187L, E189R, K190E, K196T, and F200L relative to SEQ ID NO: 1. In some embodiments, the S. aureus SrtA variant may comprise D124G, Y187L, E189R, and F200L and optionally further comprises one or more mutations of P94S/R, D160N, D165A, K190E, and K196T and optionally further comprises E105K and/or E108A relative to SEQ ID NO: 1. In certain embodiments, the S. aureus SrtA variant may comprise, relative to SEQ ID NO: 1, D124G; D124G and F200L; P94S/R, D124G, D160N, D165A, K190E, and K196T; P94S/R, D160N, D165A, Y187L, E189R, K190E, and K196T; P94S/R, D124G, D160N, D165A, Y187L, E189R, K190E, and K196T; D124G, Y187L, E189R, and F200L; or P94S/R, D124G, D160N, D165A, Y187L, E189R, K190E, K196T, and F200L. In some embodiments, mutations E105K and/or E108A/Q allows the sortase-mediated reaction to be Ca independent. In some embodiments, the S. aureus SrtA variants as described herein may have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a full length of a wild type S. aureus SrtA, e.g., as shown in SEQ ID NO: 1.

In some embodiments, a functional variant of S. aureus SrtA useful in the present disclosure may be a S. aureus SrtA variant comprising one or more mutations of P94S/R, E105K, E108A/Q, D124G, D160N, D165A, Y187L, E189R, K190E, K196T, and F200L. In certain embodiments, the S. aureus SrtA variant may comprise P94S/R, E105K, E108Q, D124G, D160N, D165A, Y187L, E189R, K190E, K196T, and F200L; or P94S/R, E105K, E108A, D124G, D160N, D165A, Y187L, E189R, K190E, K196T, and F200L. In some embodiments, the S. aureus SrtA variant may comprise one or more mutations of P94S/R, E105K, E108A/Q, D124G, D160N, D165A, Y187L, E189R, K190E, K196T, and F200L relative to SEQ ID NO: 1. In certain embodiments, the S. aureus SrtA variant may comprise P94S/R, E105K, E108Q, D124G, D160N, D165A, Y187L, E189R, K190E, K196T, and F200L relative to SEQ ID NO: 1; or P94S/R, E105K, E108A, D124G, D160N, D165A, Y187L, E189R, K190E, K196T, and F200L relative to SEQ ID NO: 1. In some embodiments, the S. aureus SrtA variants have 25-60 (e.g., 25, 30, 35, 40, 45, 50, 55, 56, 57, 58, 59, or 60) amino acids being removed from N-terminus. In some embodiments, the mutated amino acid positions above are numbered according to the numbering of a wild type S. aureus SrtA, e.g., as shown in SEQ ID NO: 1.

In some embodiments, the present disclosure contemplates a S. aureus SrtA variant (mg SrtA) comprising or consisting essentially of or consisting of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9%, or higher) identity to an amino acid sequence as set forth in SEQ ID NO: 3. In some embodiments, SEQ ID NO: 3 is a truncated SrtA and the mutations corresponding to wild type SrtA are shown in bold and underlined below. In some embodiments, the SrtA variant comprises, or consists essentially of, or consists of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9%, or higher) identity to an amino acid sequence as set forth in SEQ ID NO: 3 and comprises the mutations of P94R/S, D124G, D160N, D165A, Y187L, E189R, K190E, K196T, and F200L and optionally E105K and/or E108A/Q (numbered according to the numbering of SEQ ID NO: 1).

| (mutations shown in bold and underlined) | |
|---|---|
| SEQ ID NO: 3 | |
| 1 | KPHIDNYLHD KDKDEKIEQY DKNVKEQASK DKKQQAKPQI PKDKSKVAGY |
| 51 | IEIPDADIKE PVYPGPATRE QLNRGVSFAE ENESLDDQNI SIAGHTFIGR |
| 101 | PNYQFTNLKA AKKGSMVYFK VGNETRKYKM TSIRNVKPTA VGVLDEQKGK |
| 151 | DKQLTLITCD DLNRETGVWE TRKILVATEV K |

In some embodiments, the present disclosure provides a nucleic acid encoding the S. aureus SrtA variant, and in some embodiments the nucleic acid is set forth in SEQ ID NO: 4.

SEQ ID NO: 4
AAACCACATATCGATAATTATCTTCACGATAAAGATAAAGATGAAAAGA
TTGAACAATATGATAAAAATGTAAAAGAACAGGCGAGTAAAGATAAAAA
GCAGCAAGCTAAACCTCAAATTCCGAAAGATAAATCGAAAGTGGCAGGC
TATATTGAAATTCCAGATGCTGATATTAAAGAACCAGTATATCCAGGAC
CAGCAACACGTGAACAATTAAATAGAGGTGTAAGCTTTGCAGAAGAAAA
TGAATCACTAGATGATCAAAATATTTCAATTGCAGGACACACTTTCATT
GGCCGTCCGAACTATCAATTTACAAATCTTAAAGCAGCCAAAAAAGGTA
GTATGGTGTACTTTAAAGTTGGTAATGAAACACGTAAGTATAAAATGAC
AAGTATAAGAAATGTTAAGCCTACAGCTGTAGGAGTTCTAGATGAACAA
AAAGGTAAAGATAAACAATTAACATTAATTACTTGTGATGATCTTAATC
GGGAGACAGGCGTTTGGGAAACACGTAAAATCTTGGTAGCTACAGAAGT
CAAA

In some embodiments, the S. aureus SrtA variant can be a truncated variant with, e.g., 25-60 (e.g., 30, 35, 40, 45, 50, 55, 59, or 60) amino acids being removed from the N-terminus (as compared to the wild type S. aureus SrtA). In some embodiments, the truncated variant comprises, or consists essentially of, or consists of an amino acid sequence having at least 60% (e.g., at least 65%, 70%, 75%, 80%, 85%, 90%, 91%, 92%, 93%, 94%, 95%, 96%, 97%, 98%, 99%, 99.5%, 99.9%, or higher, such as 100%) identity to an amino acid sequence as set forth in SEQ ID NO: 5 or 7. The nucleic acids encoding SEQ ID NOs: 5 and 7 are set forth in SEQ ID NOs: 6 and 8 below.

(mutations as compared to wt SrtA being shown in bold and underlined)
SEQ ID NO: 5
QAKPQI PKDKSKVAGY
IEIPDADIKE PVYPGPATRE QLNRGVSFAE ENESLDDQNI SIAGHTFIGR
PNYQFTNLKA AKKGSMVYFK VGNETRKYKM TSIRNVKPTA VGVLDEQKGK
DKQLTLITCD DLNRETGVWE TRKILVATEV K
SEQ ID NO: 6
CAAGCTAAACCTCAAATTCCGAAAGATAAATCGAAAGTGGCAGGCTATATTGAA
ATTCCAGATGCTGATATTAAAGAACCAGTATATCCAGGACCAGCAACACGTGAA
CAATTAAATAGAGGTGTAAGCTTTGCAGAAGAAAATGAATCACTAGATGATCAA
AATATTTCAATTGCAGGACACACTTTCATTGGCCGTCCGAACTATCAATTTACAA
ATCTTAAAGCAGCCAAAAAAGGTAGTATGGTGTACTTTAAAGTTGGTAATGAAA
CACGTAAGTATAAAATGACAAGTATAAGAAATGTTAAGCCTACAGCTGTAGGAG
TTCTAGATGAACAAAAAGGTAAAGATAAACAATTAACATTAATTACTTGTGATG
ATCTTAATCGGGAGACAGGCGTTTGGGAAACACGTAAAATCTTGGTAGCTACAG
AAGTCAAA
(mutations as compared to wt SrtA being shown in bold and underlined)
SEQ ID NO: 7
MQAKPQI PKDKSKVAGY
IEIPDADIKE PVYPGPATRE QLNRGVSFAE ENESLDDQNI SIAGHTFIGR
PNYQFTNLKA AKKGSMVYFK VGNETRKYKM TSIRNVKPTA VGVLDEQKGK
DKQLTLITCD DLNRETGVWE TRKILVATEV K
SEQ ID NO: 8
ATGCAAGCTAAACCTCAAATTCCGAAAGATAAATCGAAAGTGGCAGGCTATATT
GAAATTCCAGATGCTGATATTAAAGAACCAGTATATCCAGGACCAGCAACACGT
GAACAATTAAATAGAGGTGTAAGCTTTGCAGAAGAAAATGAATCACTAGATGAT
CAAAATATTTCAATTGCAGGACACACTTTCATTGGCCGTCCGAACTATCAATTTA
CAAATCTTAAAGCAGCCAAAAAAGGTAGTATGGTGTACTTTAAAGTTGGTAATG
AAACACGTAAGTATAAAATGACAAGTATAAGAAATGTTAAGCCTACAGCTGTAG
GAGTTCTAGATGAACAAAAAGGTAAAGATAAACAATTAACATTAATTACTTGTG
ATGATCTTAATCGGGAGACAGGCGTTTGGGAAACACGTAAAATCTTGGTAGCTA
CAGAAGTCAAA

In some embodiments, a sortase A variant may comprise any one or more of the following: an S residue at position 94 (S94) or an R residue at position 94 (R94), a K residue at position 105 (K105), an A residue at position 108 (A108), a Q residue at position 108 (Q 108), a G residue at position 124 (G124), an N residue at position 160 (N160), an A residue at position 165 (A165), an R residue at position 189 (R189), an E residue at position 190 (E190), a T residue at position 196 (T196), and an L residue at position 200 (L²⁰⁰) (numbered according to the numbering of a wild type SrtA, e.g., SEQ ID NO: 1), optionally with about 25-60 (e.g., 25, 30, 35, 40, 45, 50, 55, 56, 57, 58, 59, or 60) amino acids being removed from N-terminus of the wild type S. aureus SrtA. For example, in some embodiments a sortase A variant comprises two, three, four, or five of the afore-mentioned mutations relative to a wild type S. aureus SrtA (e.g., SEQ ID NO: 1). In some embodiments a sortase A variant comprises an S residue at position 94 (S94) or an R residue at position 94 (R94), and also an N residue at position 160 (N160), an A residue at position 165 (A165), and a T residue at position 196 (T196) relative to a wild type S. aureus SrtA (e.g., SEQ ID NO: 1). For example, in some embodiments, a sortase A variant comprises P94S or P94R, and also D160N, D165A, and K196T relative to a wild type S. aureus SrtA (e.g., SEQ ID NO: 1). In some embodiments a sortase A variant comprises an S residue at position 94 (S94) or an R residue at position 94 (R94), and also an N residue at position 160 (N160), an A residue at position 165 (A165), an E residue at position 190, and a T residue at position 196 relative to a wild type S. aureus SrtA (e.g., SEQ ID NO: 1). For example, in some embodiments a sortase A variant comprises P94S or P94R, and also D160N, D165A, K190E, and K196T relative to a wild type S. aureus SrtA (e.g., SEQ ID NO: 1). In some embodiments a sortase A variant comprises an R residue at position 94 (R94), an N residue at position 160 (N160), an A residue at position 165 (A165), an E residue at position 190, and a T residue at position 196 relative to a wild type S. aureus SrtA (e.g., SEQ ID NO: 1). In some embodiments a sortase comprises P94R, D160N, D165A, K190E, and K196T relative to a wild type S. aureus SrtA (e.g., SEQ ID NO: 1). In some embodiments, the S. aureus SrtA variants may have 25-60 (e.g., 25, 30, 35, 40, 45, 50, 55, 56, 57, 58, 59, or 60) amino acids being removed from N-terminus.

In some embodiments, a sortase A variety having higher transamidase activity than a naturally occurring sortase A may be used. In some embodiments the activity of the sortase A variety is at least about 10, 15, 20, 40, 60, 80, 100, 120, 140, 160, 180, or 200 times as high as that of wild type S. aureus sortase A. In some embodiments such a sortase variant is used in a composition or method of the present disclosure. In some embodiments a sortase variant comprises any one or more of the following substitutions relative to a wild type S. aureus SrtA: P94S/R, E105K, E108A, E108Q, D124G, D160N, D165A, Y187L, E189R, K190E, K196T, and F200L mutations. In some embodiments, the SrtA variant may have 25-60 (e.g., 30, 35, 40, 45, 50, 55, 59 or 60) amino acids being removed from N-terminus.

In some embodiments, the amino acid mutation positions are determined by an alignment of a parent S. aureus SrtA (from which the S. aureus SrtA variant as described herein is derived) with the polypeptide of SEQ ID NO: 1, i.e., the polypeptide of SEQ ID NO: 1 is used to determine the corresponding amino acid sequence in the parent S. aureus SrtA. Methods for determining an amino acid position corresponding to a mutation position as described herein is well known in the art. Identification of the corresponding amino acid residue in another polypeptide can be confirmed by using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. Based on above well-known computer programs, it is routine work for those skilled in the art to determine the amino acid position of a polypeptide of interest as described herein.

In some embodiments, the sortase variant may further comprise 1, 2, 3, 4, 5, 6, 7, 8, 9, 10, 11, 12, 13, 14, or 15 conservative amino acid mutations. Conservative amino acid mutations that will not substantially affect the activity of a protein are well known in the art.

Using sortase, e.g., mg SrtA to covalently label proteins onto cells has broad prospects in scientific research and clinical applications. However, this may have certain constraints: different types of cells have different types of membrane proteins, and the number of proteins containing N-terminal glycine(s) (e.g., G1 for mg SrtA) is also different. The strategy of the present disclosure allows the possibility of using various sortases to modify various cells with agents.

### Irreversible Linkers

Since a SrtA-mediated protein-cell conjugation is a reversible reaction, to improve the efficiency of cell labeling, it would be beneficial to minimize the occurrence of reverse reactions. One solution to increase the product yield is to increase the concentration of the reaction substrates, but it may be difficult to achieve a very high concentration for macromolecular proteins in practical applications; and even if the high concentration could be reached, the high cost may limit the use of this technology. Another solution is to continuously remove the products from the reaction system so that the reaction will not stop due to equilibrium, but since the reaction is carried out on the cell, product separation may be difficult. The inventors of the present application found that surprisingly for cell labelling, the reverse reaction can be prevented by introducing a hydroxyacetyl-like byproduct that is not a substrate for the reverse reaction, thus rendering the labeling reaction irreversible.

To obtain a hydroxyacetyl-like byproduct, the present disclosure contemplates using a sortase recognition motif comprising an unnatural amino acid, preferably located at position 5 from the direction of N-terminal to C-terminal of the sortase recognition motif. In some embodiments, the unnatural amino acid is a substituted or unsubstituted hydroxyl carboxylic acid having a formulae of CH₂OH—(CH₂)ₙ—COOH, with n being an integer from 0 to 5, e.g., 0, 1, 2, 3, 4 and 5, preferably with n=0. In some embodiments, the sortase recognition motif comprising an unnatural amino acid may be selected from the group consisting of LPXT*Y, LPXA*Y, LPXS*Y, LPXL*Y, LPXV*Y, LGXT*Y, LAXT*Y, LSXT*Y, NPXT*Y, MPXT*Y, IPXT*Y, SPXT*Y, VPXT*Y, and YPXR*Y, wherein * represents the optionally substituted hydroxyl carboxylic acid; and X and Y independently represent any amino acid. In some embodiments, the sortase recognition motif comprising an unnatural amino acid may be selected from a group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein M preferably is LPET*G with * preferably being 2-hydroxyacetic acid. In some embodiments, Leu-Pro-Glu-Thr-2-hydroxyacetic acid-Gly (LPET-(2-hydroxyacetic acid)-G) is used as a linker to ensure that the byproduct would make the reaction irreversible.

To introduce the irreversible linker to an agent, in some embodiments, the sortase recognition motif comprising an unnatural amino acid as a linker is chemically synthesized and can be directly conjugated to an agent such as a protein or polypeptide.

In some embodiments, the sortase recognition motif comprising an unnatural amino acid can be conjugated to an agent by various chemical means to generate a desired sortase substrate. These methods may include chemical conjugation with a bifunctional crosslinker or a bifunctional cross-linking agent such as, e.g., an NHS ester-maleimide heterobifunctional crosslinker to connect a primary amine group with a reduced thiol group. Other molecular fusions may be formed between the sortase recognition motif and the agent, for example through a spacer. As used herein, in some embodiments, the term “spacer” refers to the residual part of a bifunctional crosslinker after crosslinking the sortase recognition motif and the agent.

Various chemical conjugation means for attaching the bifunctional crosslinker or spacer can be used in the present disclosure, including but not limited to: (1) zero-length type (e.g., EDC; EDC plus sulfo NHS; CMC; DCC; DIC; N,N′-carbonyldiimidazole; Woodward's reagent K); (2) amine-sulfhydryl type such as an NHS ester-maleimide heterobifunctional crosslinker; Maleimido carbonic acid (C₂₋₈) (e.g., 6-Maleimidohexanoic acid and 4-Maleimidobutyric acid); EMCS; SPDP, LC-SPDP, sulfo-LC-SPDP; SMPT and sulfo-LC-SMPT; SMCC, LC-SMCC, and sulfo-SMCC; MBS and sulfo-MBS; SIAB and sulfo-SIAB; SMPB and sulfo-SMPB; GMBS and sulfo-GMBS; SIAX and SIAXX; SIAC and SIACX; NPIA; (3) homobifunctional NHS esters type (e.g., DSP; DTSSP; DSS; DST and Sulfo-DST; BSOCOES and Sulfo-BSOCOES; EGS and Sulfo-EGS); (4) homobifunctional imidoesters type (e.g., DMA; DMP; DMS; DTBP); (5) carbonyl-sulfydryl type (e.g., KMUH; EMCH; MPBH; M2C2H; PDPH); (6) sulfhydryl reactive type (e.g., DPDPB; BMH; HBVS); (7) sulfhydryl-hydroxy type (e.g., PMPI); or the like.

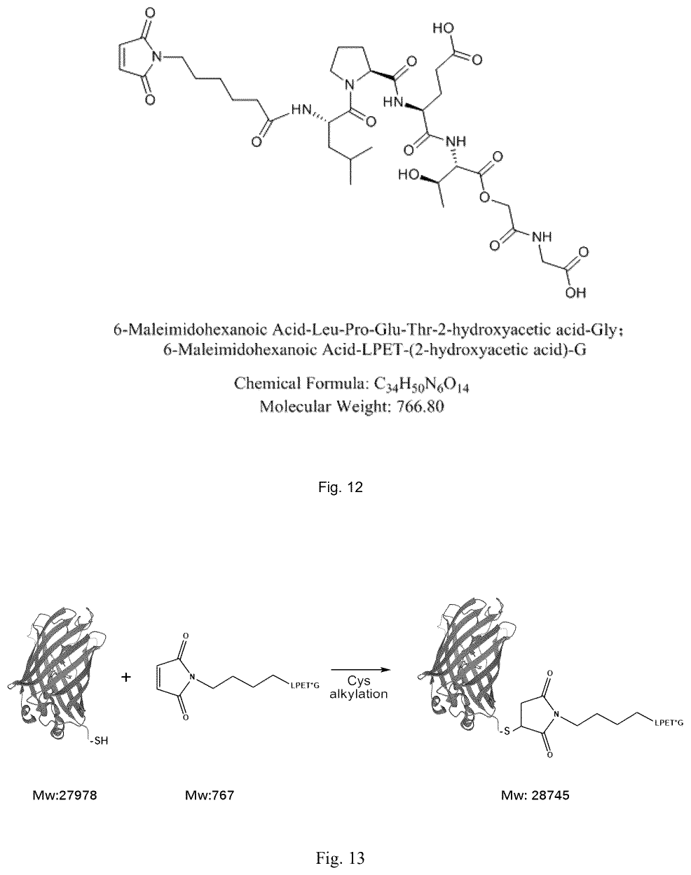

In some embodiments, an amine-sulfhydryl type or an NHS ester-maleimide heterobifunctional crosslinker is a preferred spacer that can be used herein. In certain embodiments, a maleimido carbonic acid (C₂₋₈) heterobifunctional crosslinker such as 6-Maleimidohexanoic acid and 4-Maleimido butyric acid are particularly useful spacers for the construction of desired sortase substrates. The maleimido carbonic acid (C₂₋₈) heterobifunctional crosslinker, such as 6-Maleimidohexanoic acid and 4-Maleimido butyric acid, can undergo a Michael addition reaction with an exposed sulfhydryl group, e.g., on an exposed cysteine, but this reaction will not occur with an unexposed cysteine. In one embodiment, 6-Maleimidohexanoic acid was introduced in the irreversible linker of the present disclosure, to obtain 6-Maleimidohexanoic acid-Leu-Pro-Glu-Thr-2-hydroxyacetic acid-Gly as shown in FIG. 12.

An illustrative reaction scheme for conjugation of the irreversible linker 6-Mal-LPET*G to a modified protein is shown in FIG. 13. The two reaction substrates were mixed and reacted in a ratio of 1:4=eGFP-cys:6-Mal-LPET*G to obtain the final reaction product.

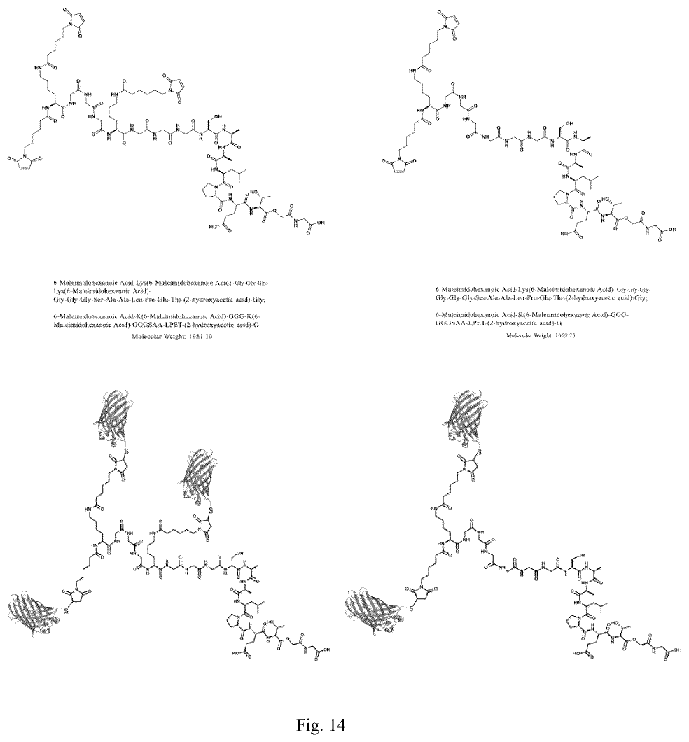

By using the spacers as described herein, especially maleimido carbonic acid (C₂₋₈) heterobifunctional crosslinkers, such as 6-Maleimidohexanoic acid and 4-Maleimido butyric acid, the inventors successfully designed linkers with different structures, including double forks, triple forks, and multiple forks. These different linkers can be used to label cells such as RBCs according to actual needs, for example to obtain multi-modal therapeutics. In the multi-fork structure design of some embodiments, one or more spacers can be linked to the amino group of the N-terminal amino acid and/or the amino group of the side chain of lysine, and the same or different agents like proteins or polypeptides can be linked to the one or more spacers, as shown in FIG. 14. This technology could further expand the variety of agents, like proteins, for cell labeling and improve the efficiency of RBC engineering.

### Sortase Substrates

Substrates suitable for a sortase-mediated conjugation can readily be designed. A sortase substrate may comprises a sortase recognition motif and an agent. For example, an agent such as a polypeptide can be modified to include a sortase recognition motif at or near the C-terminus, thereby allowing it to serve as a substrate for sortase. The sortase recognition motif need not be positioned at the very C-terminus of a substrate but should typically be sufficiently accessible by the enzyme to participate in the sortase reaction. In some embodiments, a sortase recognition motif is considered to be “near” a C-terminus if there are no more than 5, 6, 7, 8, 9, or 10 amino acids between the most N-terminal amino acid in the sortase recognition motif (e.g., L) and the C-terminal amino acid of the polypeptide. A polypeptide comprising a sortase recognition motif may be modified by incorporating or attaching any of a wide variety of moieties (e.g., peptides, proteins, compounds, nucleic acids, lipids, small molecules, and sugars) thereto.

In some embodiments, the present disclosure provides a sortase substrate comprising a structure of A¹-Sp-M, in which A¹ represents an agent, Sp represents one or more optional spacers, and M represents a sortase recognition motif comprising an unnatural amino acid as set forth herein. In some embodiments, the one or more Sp is selected from the group consisting of the following types of crosslinkers: (1) zero-length type; (2) amine-sulfhydryl type; (3) homobifunctional NHS esters type; (4) homobifunctional imidoesters type; (5) carbonyl-sulfydryl type; (6) sulfhydryl reactive type; and (7) sulfhydryl-hydroxy type; preferably the one or more Sp is a maleimido carbonic acid (C₂₋₈) type heterobifunctional crosslinker, such as 6-Maleimidohexanoic acid and 4-Maleimidobutyric acid, and the agent comprises an exposed sulfydryl, preferably an exposed cysteine, more preferably a terminal cysteine, most preferably a C-terminal cysteine. In some embodiments, when two or more spacers are present, the agents linked to the spacers can be the same or different.

### Agents

Depending on the intended applications of the modified cells, a wide variety of agents such as a binding agent, a therapeutic agent, or a detection agent can be contemplated in the present disclosure. In some embodiments, an agent may comprise a protein, a peptide (e.g., an extracellular domain of oligomeric ACE2), an antibody (e.g., an anti-PD1 antibody) or its functional antibody fragment, an antigen or epitope, a MHC-peptide complex, a drug such as a small molecule drug (e.g., an antitumor agent such as a chemotherapeutic agent), an enzyme (e.g., a functional metabolic or therapeutic enzyme), a hormone, a cytokine, a growth factor, an antimicrobial agent, a probe, a ligand, a receptor, an immunotolerance-inducing peptide, a targeting moiety, or any combination thereof.

In some embodiments, in addition to a therapeutically active domain such as an enzyme, a drug, a small molecule (such as a small molecule drug (e.g., an antitumor agent such as a chemotherapeutic agent)), a therapeutic protein and a therapeutic antibody as described herein, the agent may further comprise a targeting moiety for targeting the cells and/or agent to a site in the body where the therapeutic activity is desired. In some embodiments, the targeting moiety binds to a target present at such a site. Any targeting moiety may be used, e.g., an antibody. The site may be any organ or tissue, e.g., respiratory tract (e.g., lung), bone, kidney, liver, pancreas, skin, cardiovascular system (e.g., heart), smooth or skeletal muscle, gastrointestinal tract, eye, blood vessel surfaces, etc.

In some embodiments, a protein is an enzyme such as a functional metabolic or therapeutic enzyme, e.g., an enzyme that plays a role in metabolism or other physiological processes in a mammal. In some embodiments a protein is an enzyme that plays a role in carbohydrate metabolism, amino acid metabolism, organic acid metabolism, porphyrin metabolism, purine or pyrimidine metabolism, and/or lysosomal storage. Deficiencies of enzymes or other proteins can lead to a variety of diseases, e.g., diseases associated with defects in carbohydrate metabolism, amino acid metabolism, organic acid metabolism, purine or pyrimidine metabolism, lysosomal storage disorders, and blood clotting, among others. Metabolic diseases are characterized by the lack of functional enzymes or excessive intake of metabolites. Thus, the metabolite's deposition in the circulation and tissues causes tissue damage. Due to the wide distribution of blood cells such as RBCs in the human body, the present disclosure contemplates modifying membrane proteins of blood cells such as RBCs with functional metabolic enzymes. The enzyme-targeted blood cells, such as RBCs, will uptake metabolites in the plasma of patients. Exemplary enzymes include urate oxidase for gout, phenylalanine ammonia-lyase for Phenylketonuria, acetaldehyde dehydrogenase for alcoholic hepatitis, butyrylcholinesterase for cocaine metabolite, and the like. In some embodiments, red blood cells having urate oxidase conjugated thereto may be administered to a subject in need of treatment of chronic hyperuricemia, e.g., a patient with gout, e.g., gout that is refractory to other treatments.

Enzyme replacement therapy has been a specific treatment for patients with, e.g., lysosomal storage disorders (LSDs) over the past three decades. However, this medication has some limitations, such as immune system problems and financial burden. In addition, the therapeutic enzymes are rapidly cleared from the human body by extensive catabolism. In some embodiments, the present disclosure contemplates binding the therapeutic enzymes to RBC membrane proteins through the sortase reaction as described herein. The use of blood cells, such as RBCs, as carriers will target the functional enzymes to macrophages in liver, where blood cells such as RBCs are cleared, and also reduce the dosage and frequency of drug interventions for the enhanced half-time of enzymes. Exemplary enzymes include glucocerebrosidase for Gaucher disease, α-galactosidase for Fabry disease, and alanine glycoxylate aminotransferase and glyoxylate reductase/hydroxypyruvate reductase for primary hyperoxaluria.

In some embodiments, the agent may comprise a peptide. Various functional peptides can be contemplated in the present disclosure. In certain embodiments, the peptide may comprise an oligomeric ACE2 extracellular domain.

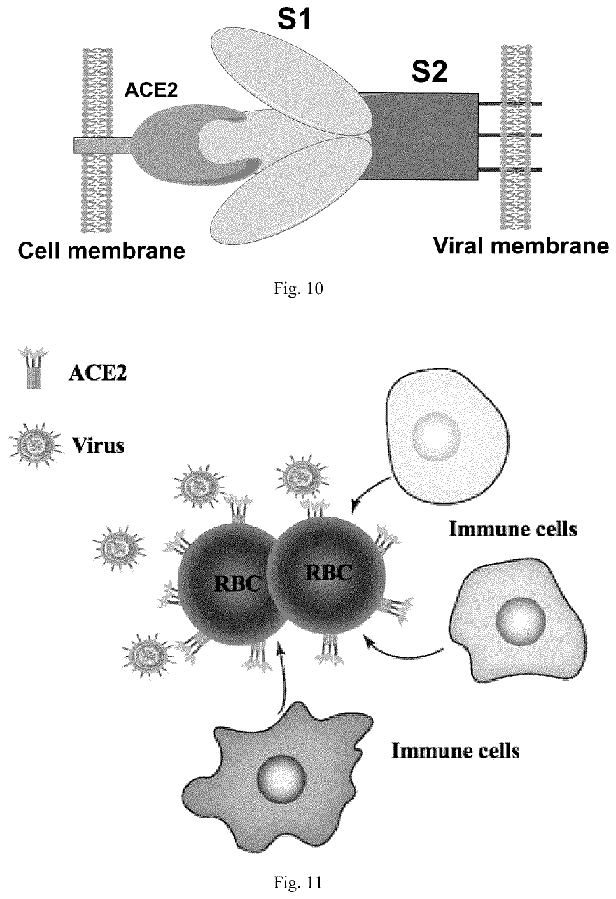

SARS-CoV-2, which causes a respiratory disease named COVID-19, belongs to the same coronaviridea as SARS-CoV. The genome of SARS-CoV-2 is very similar to SARS-CoV sharing ˜80% nucleotide sequence identity and 94.6% amino acid sequence identity in the ORF encoding the spike protein. SARS-CoV-2 and SARS-CoV spike proteins have very similar structures, both entering human cells through spike protein interaction with ACE2 as shown in FIG. 10. Unfortunately, seventeen years after the SARS pandemic, no effective detection (except RT-PCR), prevention, or treatment approaches were developed from SARS-CoV that could be readily applied to SARS-CoV-2. This has caught everybody in a hurry to come up with different strategies, including SARS-CoV-2 specific antibodies, vaccines, protease inhibitors, and RNA-dependent RNA polymerase inhibitors to detect and combat the SARS-CoV-2 associated disease “COVID-19.” These efforts may be useful for SARS-CoV-2 if developed quickly enough (probably within 2-3 months). However, they still may not be applied to future coronaviruses given the fact that RNA viruses have a high mutation rate. The lack of cross-reactivity between several SARS-CoV specific antibodies and SARS-CoV-2 is a clear demonstration of this. Thus, detection devices or therapeutic agents that are not only useful for SARS-CoV-2, but also could be readily applied to future coronavirus are highly desirable for development.

Both SARS-CoV and SARS-CoV-2 enter host cells through binding with ACE2 by the viral S protein. This mechanism is also applied by other coronavirus in order to successfully establish infection. Thus, molecules blocking the S protein interaction with ACE2 could prevent virus infection. It has been shown that the ACE2 extracellular domain can block virus infection. However, monomeric ACE2 only has limited binding affinity to S protein and is not expected to have a high virus blocking activity. High-affinity oligomeric ACE2 on the other hand possesses a high virus binding affinity and could effectively compete with cell surface ACE2 for virus neutralization.

Cell assays have demonstrated that coronavirus infection or even S protein binding with ACE2 will cause shedding of ACE2 from the cell surface, resulting in decreased cell surface ACE2 expression levels[4][5]. Down regulation of ACE2 results in angiotensin II accumulation which is closely related with acute lung injury[4][6][7]. This perhaps could explain the fact that coronavirus infected patients show respiratory syndromes, especially in the lung. The fact that coronavirus infected patients show respiratory syndromes and some even develop ARDS suggests supplementing ACE2 could also alleviate respiratory syndromes for virus infection treatment.

In some embodiments, the present disclosure contemplates using blood cells, such as red blood cells, as oligomeric ACE2 carriers for effective virus neutralization (FIG. 11), by use of the new strategy to modify natural blood cells, such as RBCs, with peptides and/or small molecules through a sortase mediated reaction as described herein.

In some embodiments, the agent may comprise an antibody, including an antibody, an antibody chain, an antibody fragment, e.g., scFv, an antigen-binding antibody domain, a VHH domain, a single-domain antibody, a camelid antibody, a nanobody, an adnectin, or an anticalin. The red blood cells having antibodies attached thereto may be used as a delivery vehicle for the antibodies and/or the antibodies may serve as a targeting moiety. Exemplary antibodies include anti-tumor antibodies such as PD-1 antibodies, e.g., Nivolumab and Pembrolizumab, which both are monoclonal antibodies for human PD-1 protein and are now the forefront treatment for melanoma, non-small cell lung carcinoma, and renal-cell cancer. The heavy chains of the antibodies modified with a sortase recognition motif, such as LPETG, can be expressed and purified. In the same way, PD-L1 antibodies, such as Atezolizum, Avelumab and Durvalumab targeting PD-L1 for treating urothelial carcinoma and metastatic merkel cell carcinoma, can be modified. Also, Adalimumab, Infliximab, Sarilumab, and Golimumab, which are FDA-approved therapeutic monoclonal antibodies for curing rheumatoid arthritis, can be modified by using the method as described herein.

In some embodiments, the agent may comprise an antigen or epitopes or a binding moiety that binds to an antigen or epitope. In some embodiments an antigen is any molecule or complex comprising at least one epitope recognized by a B cell and/or by a T cell. An antigen may comprise a polypeptide, a polysaccharide, a carbohydrate, a lipid, a nucleic acid, or combination thereof. An antigen may be naturally occurring or synthetic, e.g., an antigen naturally produced by and/or genetically encoded by a pathogen, an infected cell, a neoplastic cell (e.g., a tumor or cancer cell), a virus, a bacteria, a fungus, or a parasite. In some embodiments, an antigen is an autoantigen or a graft-associated antigen. In some embodiments, an antigen is an envelope protein, capsid protein, secreted protein, structural protein, cell wall protein or polysaccharide, capsule protein or polysaccharide, or enzyme. In some embodiments an antigen is a toxin, e.g., a bacterial toxin. An antigen or epitope may be modified, e.g., by conjugation to another molecule or entity (e.g., an adjuvant).

In some embodiments, red blood cells having an epitope, antigen, or portion thereof conjugated thereto as described herein may be used as vaccine components. In some embodiments an antigen conjugated to red blood cells as described herein may be any antigen used in a conventional vaccine known in the art.

In some embodiments an antigen is a surface protein or polysaccharide of, e.g., a viral capsid, envelope, or coat, or a bacterial, fungal, protozoal, or parasite cell. Exemplary viruses may include, e.g., coronaviruses (e.g., SARS-CoV and SARS-CoV-2), HIV, dengue viruses, encephalitis viruses, yellow fever viruses, hepatitis virus, Ebola viruses, influenza viruses, and herpes simplex virus (HSV) 1 and 2.

In some embodiments an antigen is a tumor antigen (TA), which can be any antigenic substance produced by cells in a tumor, e.g., tumor cells or in some embodiments tumor stromal cells (e.g., tumor-associated cells such as cancer-associated fibroblasts or tumor-associated vasculature).

In some embodiments, an antigen is a peptide. Peptides may bind directly to MHC molecules expressed on cell surfaces, may be ingested and processed by APC and displayed on APC cell surfaces in association with MHC molecules, and/or may bind to purified MHC proteins (e.g., MHC oligomers). In some embodiments a peptide contains at least one epitope capable of binding to an appropriate MHC class I protein and/or at least one epitope capable of binding to an appropriate MHC class II protein. In some embodiments a peptide comprises a CTL epitope (e.g., the peptide can be recognized by CTLs when bound to an appropriate MHC class I protein).

In some embodiments, the agent may comprise a MHC-peptide complex, which may comprise a MHC and a peptide such as an antigenic peptide or an antigen as described herein for activating immune cells. In some embodiments, the antigenic peptide is associated with a disorder and is able to activate CD8⁺ T cells when presented by a MHC class I molecule. Class-I major histocompatibility complex (MHC-I) presents antigen peptides to and activates immune cells, particularly CD8⁺ T cells, which are important for fighting against cancers, infectious diseases, etc. MHC-peptide complexes with sortase recognition motifs, such as LPETG, can be expressed and purified exogenously through eukaryotic or prokaryotic systems. The purified MHC-peptide complexes will be covalently bound to blood cells, such as RBCs, by sortase-mediated reactions as described herein. In the present disclosure, the inventors used MHC-I-OT1 complex as an example. Mouse MHC-I-OT1 protein was expressed by E. coli and purified by histidine-tagged affinity chromatography. The purified MHC-I-OT1 complexes were successfully conjugated to the phospholipid incorporated in the cell membrane of RBC cells. Similarly, MHC-II presents antigen peptides to and activates immune cells, particularly CD4⁺ T cells, and thus a MHC complex comprising MHC-II and an antigen or an antigenic peptide can be bound to RBCs by sortase-mediated reactions as described herein.

This strategy of MHC complex can be used to treat or prevent diseases caused by viruses, such as HPV (targeting E6/E7), coronavirus (e.g., targeting SARS-CoV or SARS-CoV-2 Spike protein), and influenza virus (e.g., targeting H antigen/N antigen). This strategy of MHC complex can also be used to target tumor mutations, for example Kras with mutations such as V8M and/or G12D, Alk with a mutation such as E1171D, Braf with a mutation such as W487C, Jak2 with a mutation such as E92K, Stat3 with a mutation such as M28I, Trp53 with mutations such as G242V and/or S258I, Pdgfra with a mutation such as V88I, and Brca2 with a mutation such as R2066K, for tumor treatment.

In some embodiments, the agent may comprise a growth factor. In some embodiments, the agent may comprise a growth factor for one or more cell types. Growth factors include, e.g., members of the vascular endothelial growth factor (VEGF, e.g., VEGF-A, VEGF-B, VEGF-C, VEGF-D), epidermal growth factor (EGF), insulin-like growth factor (IGF; IGF-1, IGF-2), fibroblast growth factor (FGF, e.g., FGF1-FGF22), platelet derived growth factor (PDGF), or nerve growth factor (NGF) families.

In some embodiments, the agent may comprise a cytokine or the biologically active portion thereof. In some embodiments a cytokine is an interleukin (IL), e.g., any of IL-1 to IL-38 (e.g., IL-2, IL-3, IL-4, IL-5, IL-6, IL-7, IL-12), interferons (e.g., a type I interferon, e.g., IFN-α), and colony stimulating factors (e.g., G-CSF, GM-CSF, M-CSF). Cytokine (such as recombinant IL-2, recombinant IL-7, recombinant IL-12) loaded RBCs is a therapeutic delivery system for increasing tumor cytotoxicity and IFN-7 production.

In some embodiments, the agent may comprise a small molecule, e.g., those used as targeting moieties, immunomodulators, detection agents, therapeutic agents, or ligands (such as CD19, CD47, TRAIL, TGF, CD44) to activate or inhibit a corresponding receptor.

In some embodiments, the agent may comprise a receptor or receptor fragment. In some embodiments, the receptor is a cytokine receptor, growth factor receptor, interleukin receptor, or chemokine receptor. In some embodiments a growth factor receptor is a TNFα receptor (e.g., Type I TNF-α receptor), VEGF receptor, EGF receptor, PDGF receptor, IGF receptor, NGF receptor, or FGF receptor. In some embodiments a receptor is a TNF receptor, LDL receptor, TGF receptor, or ACE2.

In some embodiments, an agent to be conjugated may comprise an anti-cancer or anti-tumor agent, for example, a chemotherapy drug. In certain embodiments, cells such as red blood cells are conjugated both with an anti-tumor agent and a targeting moiety, wherein the targeting moiety targets the cells, such as red blood cells, to a cancer. Anti-cancer agents are conventionally classified in one of the following group: radioisotopes (e.g., Iodine-131, Lutetium-177, Rhenium-188, Yttrium-90), toxins (e.g., diphtheria, pseudomonas, ricin, gelonin), enzymes, enzymes to activate prodrugs, radio-sensitizing drugs, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. In some embodiments an anti-tumor agent is a protein such as a monoclonal antibody or a bispecific antibody such as anti-receptor tyrosine kinases (e.g., cetuximab, panitumumab, trastuzumab), anti-CD20 (e.g., rituximab and tositumomab) and others for example alemtuzumab, aevacizumab, and gemtuzumab; an enzyme such as asparaginase; a chemotherapy drug including, e.g., alkylating and alkylating-like agents such as nitrogen mustards; platinum agents (e.g., alkylating-like agents such as carboplatin, cisplatin), busulfan, dacarbazine, procarbazine, temozolomide, thioTEPA, treosulfan, and uramustine; purines such as cladribine, clofarabine, fludarabine, mercaptopurine, pentostatin, thioguanine; pyrimidines such as capecitabine, cytarabine, fluorouracil, floxuridine, gemcitabine; cytotoxic/anti-tumor antibiotics such anthracyclines (e.g., daunorubicin, doxorubicin, epirubicin, idarubicin, mitoxantrone, pixantrone, and valrubicin); and others for example taxol, nocodazole, or β-Ionone. Antitumor agent loaded cells such as RBCs via phospholipid anchor plus sortase-mediated reactions are promising for decreasing antibiotic toxicity and increasing circulation times and can perform as a slow drug delivery.

In some embodiments, a tumor is a malignant tumor or a “cancer.” The term “tumor” includes malignant solid tumors (e.g., carcinomas, sarcomas) and malignant growths with no detectable solid tumor mass (e.g., certain hematologic malignancies). The term “cancer” is generally used interchangeably with “tumor” herein and/or to refer to a disease characterized by one or more tumors, e.g., one or more malignant or potentially malignant tumors. Cancer includes, but is not limited to: breast cancer; biliary tract cancer; bladder cancer; brain cancer; cervical cancer; choriocarcinoma; colon cancer; endometrial cancer; esophageal cancer; gastric cancer; hematological neoplasms; T-cell acute lymphoblastic leukemia/lymphoma; hairy cell leukemia; chronic lymphocytic leukemia; chronic myelogenous leukemia; multiple myeloma; adult T-cell leukemia/lymphoma; intraepithelial neoplasms; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells, and mesenchymal cells; neuroblastoma; pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer; testicular cancer; and thyroid cancer.

In some embodiments, an agent to be conjugated may comprise an anti-microbial agent. An anti-microbial agent may include compounds that inhibit proliferation or activity of, destroy, or kill bacteria, viruses, fungi, or parasites. In some embodiments the red blood cells are conjugated with an anti-microbial agent against a bacteria, virus, fungi, or parasite and with a targeting moiety, wherein the targeting moiety targets the cell to the bacteria, virus, fungi, or parasite. In some embodiments, the anti-microbial agent may include β-lactamase inhibitory proteins or metallo-beta-lactamase for treating bacterial infections.

In some embodiments, an agent to be conjugated may comprise probes, which can be used as, for example, diagnostic tools. Molecular imaging has been demonstrated as an efficient way for tracking disease progression such as in cancer. Small molecular probes such as fluorescein can be labeled on cells through the methods as described herein, instead of conventional chemical reaction which may cause damage to cells.

In some embodiments, an agent to be conjugated may comprise a prodrug. The term “prodrug” refers to a compound that, after in vivo administration, is metabolized or otherwise converted to the biologically, pharmaceutically, or therapeutically active form of the compound. A prodrug may be designed to alter the metabolic stability or the transport characteristics of a compound, to mask side effects or toxicity, to improve the flavor of a compound, and/or to alter other characteristics or properties of a compound. By virtue of knowledge of pharmacodynamic processes and drug metabolisms in vivo, once a pharmaceutically active compound is identified, those skilled in the pharmaceutical arts can design prodrugs of the compound (Nogrady, “Medicinal Chemistry A Biochemical Approach,” 1985, Oxford University Press: N.Y., pages 388-392). Procedures for the selection and preparation of suitable prodrugs are also known in the art. In the context of the present invention, a prodrug is preferably a compound that, after in vivo administration, is converted to its active form via enzymatic catalysis.

### Methods for Modifying Cells

Sortase can recognize specific sortase recognition motifs, like sequence LPXTG, and connect the LPXTG at the C-terminus of a protein with the G at the N-terminus of another protein through a transpeptidation reaction. This principle can be used to modify an agent of interest so that the agent can be attached to a linker comprising a terminal glycine that has been linked to at least one membrane protein of a cell, such as an RBC.

In an aspect, the present disclosure provides a method for modifying a cell, comprising: (i) providing GlyₘXₙ-L², in which Glyₘ represents m glycines with m preferably being 1-5, and Xₙ represents n spacing amino acids with n preferably being 0-10, and L^{2′} represents the residual part of a first crosslinker after linking to GlyₘXₙ; (ii) treating a cell with GlyₘXₙ-L² under suitable conditions to link the GlyₘXₙ-L² to at least one membrane protein of the cell; and (iii) contacting the treated cell with a sortase substrate that comprises a sortase recognition motif and an agent, in the presence of a sortase under one or more conditions suitable for the sortase to conjugate the sortase substrate to the Glyₘ by a sortase-mediated reaction.

In some embodiments, before the treating step, a step of pre-treating the cell with a reducing agent is performed to form or increase the number of exposed sulfhydryls, when sulfhydryl is one of the reactive groups of the bifunctional crosslinker to be used. The present disclosure contemplates various reducing agents as long as they are capable of reducing the disulfide linkages within or between surface membrane proteins so as to expose sulfhydryl. In some embodiments, reducing agents that would have no or little negative effect on the viability of the treated cells are used. In some embodiments, a reducing agent such as tris(2-carboxyethyl)phosphine hydrochloride (TCEP) or dithiothreitol (DTT) or β-mercaptoethanol can be used, e.g., under partial or total reducing conditions.

It would be appreciated that those of ordinary skilled in the art are able to select conditions (e.g., optimal temperature, pH, reaction time, concentration) suitable for the sortase to attach the sortase substrate to a linker comprising a terminal glycine according to the nature of sortase substrate, the type of sortase, and the like.

It would be also appreciated that those of ordinary skilled in the art are able to select suitable conditions (e.g., optimal temperature, pH, reaction time, concentration) for linking a linker comprising a terminal glycine to at least one membrane protein of a cell, e.g., blood cells such as RBCs.

### Uses

Modified cells described herein have a number of uses. In some embodiments, the modified cells may be used as vaccine components, delivery systems, or diagnostic tools. In some embodiments, the modified cells may be used to treat or prevent various disorders, conditions, or diseases as described herein such as tumors or cancers, metabolic diseases such as lysosomal storage disorders (LSDs), bacterial infections, virus infections such as coronavirus for example SARS-COV or SARS-COV-2 infection, autoimmune diseases, or inflammatory diseases. In some embodiments, the modified cells may be used in cell therapy. In some embodiments, the therapy is administered for treatment of cancer, infections such as bacterial or virus infections, autoimmune diseases, or enzyme deficiencies. In some embodiments, the cells modified with peptides for inducing immunotolerances may be used to modulate an immune response such as inducing immunotolerance. In some embodiments, the administered cells may originate from the individual to whom they are administered (autologous), may originate from different genetically identical individual(s) of the same species (isogeneic), may originate from different non-genetically identical individual(s) of the same species (allogeneic), or may originate from individual(s) of a different species. In certain embodiments, allogeneic cells may originate from an individual who is immunocompatible with the subject to whom the cells are administered.

In some embodiments, the modified cells are used as a delivery vehicle or system for the agent. For example, modified cells having a protein conjugated to a phospholipid in their cell membrane may serve as delivery vehicles for the protein. Such cells may be administered to a subject suffering from a deficiency of the protein or who may benefit from increased levels of the protein. In some embodiments the cells are administered to the circulatory system, e.g., by infusion. Examples of various diseases associated with deficiency of various proteins, e.g., enzymes, are provided above. In some embodiments, using the modified cells as a delivery system can achieve a retention release, for example for delivering hormones like glucocorticoids, insulin, and/or growth hormones in a retention release profile.

In some embodiments, the present disclosure provides a method for diagnosing, treating or preventing a disorder, condition, or disease in a subject in need thereof, comprising administering the cell or composition as described herein to the subject. In some embodiments, the disorder, condition, or disease is selected from the group consisting of tumors or cancers, metabolic diseases such as lysosomal storage disorders (LSDs), bacterial infections, virus infections such as coronavirus for example SARS-COV or SARS-COV-2 infection, autoimmune diseases, and inflammatory diseases.

As used herein, “treating,” “treat,” or “treatment” refers to a therapeutic intervention that at least partly ameliorates, eliminates, or reduces a symptom or pathological sign of a pathogen-associated disease, disorder, or condition after it has begun to develop. Treatment need not be absolute to be beneficial to the subject. The beneficial effect can be determined using any methods or standards known to the ordinarily skilled artisan.

As used herein, “preventing,” “prevent,” or “prevention” refers to a course of action initiated prior to infection by, or exposure to, a pathogen or molecular components thereof and/or before the onset of a symptom or pathological sign of the disease, disorder, or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder, or condition, or exhibits only early signs for the purpose of decreasing the risk of developing a symptom or pathological sign of the disease, disorder, or condition.

In some embodiments, the method as described herein further comprises administering the modified cells to a subject, e.g., directly into the circulatory system, e.g., intravenously, by injection or infusion.

In another aspect, a method of delivering an agent to a subject in need thereof is provided, comprising administering the modified cell or the composition as described herein to the subject. The term “delivery” or “delivering” refers to transportation of a molecule or agent to a desired cell or tissue site. Delivery can be to the cell surface, cell membrane, cell endosome, nucleus, within the cell membrane, or within the nucleus, or any other desired area of the cell.

In another aspect, a method of increasing the circulation time or plasma half-life of an agent in a subject is provided, comprising attaching the agent to a cell according to the method as described herein. In some embodiments the method further comprises administering the cell having the agent attached thereto to the subject, e.g., directly into the circulatory system, e.g., intravenously or by injection or infusion.

In some embodiments, a subject receives a single dose of cells, or receives multiple doses of cells, e.g., between 2 and 5, 10, 20, or more doses, over a course of treatment. In some embodiments a dose or total cell number may be expressed as cells/kg. For example, a dose may be about 10³, 10⁴, 10⁵, 10⁶, 10⁷, or 10⁸ cells/kg. In some embodiments a course of treatment lasts for about 1 week to 12 months or more e.g., 1, 2, 3, or 4 weeks or 2, 3, 4, 5, or 6 months. In some embodiments a subject may be treated about every 2-4 weeks. One of ordinary skilled in the art will appreciate that the number of cells, doses, and/or dosing interval may be selected based on various factors such as the weight and/or blood volume of the subject, the condition being treated, response of the subject, etc. The exact number of cells required may vary from subject to subject, depending on factors such as the species, age, weight, sex, and general condition of the subject, the severity of the disease or disorder, the particular cell(s), the identity and activity of agent(s) conjugated to the cells, mode of administration, concurrent therapies, and the like.

### Composition

In another aspect, the present disclosure provides a composition comprising the modified cell as described herein and optionally a physiologically acceptable carrier, such as in the form of a pharmaceutical composition, a delivery composition, a diagnostic composition, or a kit.

In some embodiments, the composition may comprise a plurality of cells such as blood cells, e.g., RBCs. In some embodiments, at least a selected percentage of the cells in the composition are modified, i.e., having an agent conjugated thereto by the method as described herein. For example, in some embodiments at least 5%, 10%, 15%, 20%, 25%, 30%, 40%, 50%, 60%, 70%, 80%, 90%, 95%, 96%, 97%, 98%, 99%, or more of the cells have an agent conjugated thereto. In some embodiments, two or more red blood cells or red blood cell populations conjugated with different agents are included.

In some embodiments, the composition comprises modified cells of the present disclosure, such as blood red cells, wherein the cells are modified with any agent of interest. In some embodiments, a composition comprises an effective amount of cells, e.g., up to about 10¹⁴ cells, e.g., about 10, 10², 10³, 10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×13, or 10¹⁴ cells. In some embodiments the number of cells may range between any two of the afore-mentioned numbers.

As used herein, the term “an effective amount” refers to an amount sufficient to achieve a biological response or effect of interest, e.g., reducing one or more symptoms or manifestations of a disease or condition or modulating an immune response. In some embodiments a composition administered to a subject comprises up to about 10¹⁴ cells, e.g., about 10³, 10⁴, 10⁵, 10⁶, 10⁷, 10⁸, 10⁹, 10¹⁰, 10¹¹, 10¹², 10¹³, or 10¹⁴ cells, or any intervening number or range.

As used herein, the term “a physiologically acceptable carrier” means a solid or liquid filler, diluent, or encapsulating substance that may be safely used in systemic administration. Depending upon the particular route of administration, a variety of carriers, diluents, and excipients well known in the art may be used. These may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline, and salts such as mineral acid salts including hydrochlorides, bromides and sulfates, organic acids such as acetates, propionates and malonates, water and pyrogen-free water.

It will be appreciated by those skilled in the art that other variations of the embodiments described herein may also be practiced without departing from the scope of the invention. Other modifications are therefore possible.

Although the disclosure has been described and illustrated in exemplary forms with a certain degree of particularity, it is noted that the description and illustrations have been made by way of example only. Numerous changes in the details of construction and combination and arrangement of parts and steps may be made. Accordingly, such changes are intended to be included in the invention, the scope of which is defined by the claims.

### EXAMPLES

### Example 1. Preparation of RBCs Labeling with eGFP by Using GAASK-Mal

Recombinant Protein Expression and Purification in E. coli

Mg SrtA and eGFP-LPETG cDNA were cloned in pET vectors and transformed in E. coli BL21 (DE3) cells for protein expression. Transformed cells were cultured at 37° C. until the OD₆₀₀ reached 0.6, and then 500 μM IPTG was added. The cells were cultured with IPTG for 4 hrs at 37° C. until harvested by centrifugation and subjected to lysis by precooled lysis buffer (20 mM Tris-HCl, pH 7.8, 500 mM NaCl). The lysates were sonicated on ice (5 s on, 5 s off, 60 cycles, 25% power, Branson Sonifier 550 Ultrasonic Cell Disrupter). All supernatants were filtered by 0.45 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with elution buffer containing 20 mM Tris-HCl, pH 7.8, 500 mM NaCl and 300 mM imidazole. All eluted fractions were analyzed on an SDS-PAGE gel.

An amino acid sequence of EGFP-LEPTG is as shown in SEQ ID NO: 9 below:

MHHHHHHMVSKGEELFTGVVPILVELDGDVNGHKFSVSGEGEGDATYGK
LTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPI
GDGPVLLPDNHYLSTQSALSKDPNEKRDHMVLLEFVTAAGITLGMDELY
KAALPETG

### RBC Pre-Treatment

Red blood cells were separated from peripheral blood of C57BL/6J mice by density gradient centrifugation. The separated red blood cells were washed with PBS for 3 times. Then the RBCs were pretreated with 1 mM TCEP for 1 hr at RT. Then the pretreated RBCs were washed with PBS for 3 times. GAASK-mal linker (Synthesized by Beijing Scilight Biotechnology Led. Co.) (see FIG. 2) was synthesized with more than 99% purity. GAASK-mal was dissolved in 37° C. phosphate buffer to a final concentration of 100 μM. Then 1×10⁹ RBCs were contacted with 50 μM GAASK-mal for 30 mins at 37° C. Then the obtained RBCs were washed with PBS for 3 times. The RBCs were either used immediately or stored at 4° C. for further use.

### GAASK-Mal-Mediated Labeling of RBCs

Reactions were performed in a total volume of 200 μL at RT for 2 hrs in PBS buffer while being rotated at a speed of 10 rpm. The concentration of GAASK-mal-RBCs in the reaction was 1×10⁹/mL. In the reaction system, the final concentration of mg SrtA was 10 μM and the eGFP-LEPTG substrate was 10 mM, respectively. After the reaction, the labeling efficiency of RBCs was analyzed by flow cytometry (FACS) using Beckman Coulter CytoFLEX LX.

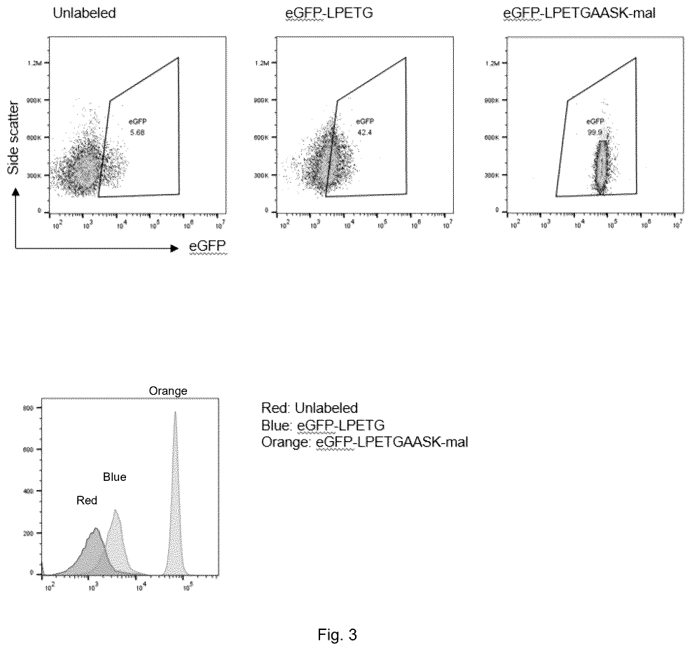

The labeling efficacy of eGFP on the RBC membrane was characterized. The results showed that about 100% of natural RBCs were eGFP-LPETGAASK-mal-labeled in vitro, the signal intensity was dose-dependent, and the labeling efficacy of eGFP-LPETGAASK-mal-labelling was about 15 times higher than the efficacy of the eGFP-LEPTG (see FIG. 3).

The Life-Span of RBCs Labeled with eGFP-LPETGAASK-Mal In Vivo

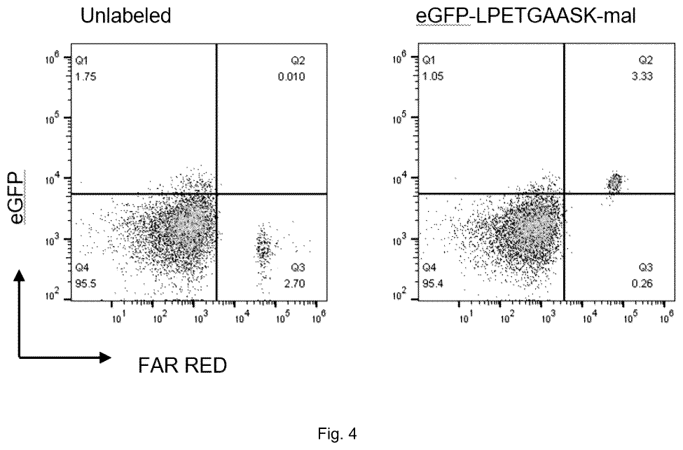

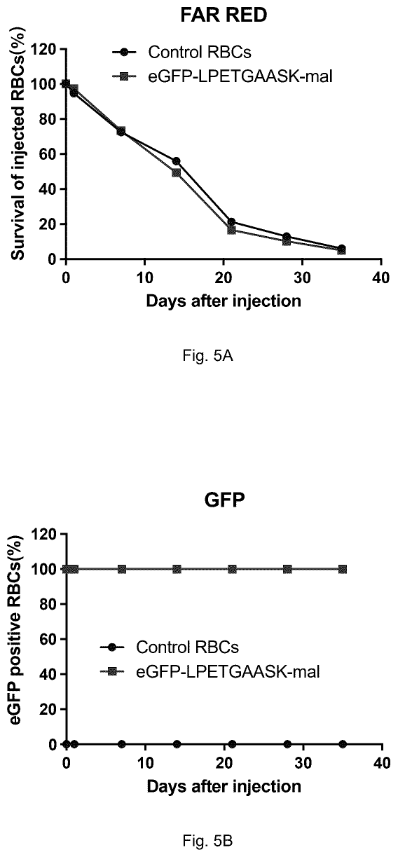

To assess the life-span of the above obtained surface modified RBCs in vivo, we next transfused the eGFP-LPETGAASK-mal labeled mouse RBCs (Dosage: 1×10⁹/mouse), which were simultaneously labeled by a fluorescent dye CellTrace Far Red according to the manufacturing protocol, into recipient mice. The percentage of Far Red positive and eGFP-LPETGAASK-mal positive RBCs in vivo was analyzed periodically through FACS. The eGFP-LPETGAASK-mal labeled RBCs not only showed the same lifespan as that of the control groups (Mice that were transfused RBCs without eGFP-LPETGAASK-mal label) but also exhibited sustained GFP signals in circulation for 28 days (see FIG. 4 and FIGS. 5A-5B).

### Example 2. Preparation of RBCs Labeled with PAL (Phenylalanine Ammonia-Lyase) by Using GAASK-Mal

Recombinant Protein Expression and Purification in E. coli

PAL-LPETG cDNA were cloned in pET vectors and transformed in E. coli BL21 (DE3) cells for protein expression. Transformed cells were cultured at 37° C. until the OD₆₀₀ reached 0.6, and then 500 μM IPTG was added. The cells were cultured with IPTG for 4 hrs at 37° C. until harvested by centrifugation and subjected to lysis by precooled lysis buffer (20 mM Tris-HCl, pH 7.8, 500 mM NaCl). The lysates were sonicated on ice (5 s on, 5 s off, 60 cycles, 25% power, Branson Sonifier 550 Ultrasonic Cell Disrupter). All supernatants were filtered by 0.45 μM filter after centrifugation at 14,000 g for 40 min at 4° C. Filtered supernatants were loaded onto HisTrap FF 1 mL column (GE Healthcare) connected to the AKTA design chromatography systems. The proteins were eluted with the elution buffer containing 20 mM Tris-HCl, pH 7.8, 500 mM NaCl and 300 mM imidazole. All eluted fractions were analyzed on an SDS-PAGE gel.

The amino acid sequence of PAL-LEPTG is as shown in SEQ ID NO: 10 below:

MHHHHHHSSGLVPRGSGMKETAAAKFERQHMDSPDLGTDDDDKAMADIG
SMHHHHHHKTLSQAQSKTSSQQFSFTGNSSANVIIGNQKLTINDVARVA
RNGTLVSLTNNTDILQGIQASCDYINNAVESGEPIYGVTSGFGGMANVA
ISREQASELQTNLVWFLKTGAGNKLPLADVRAAMLLRANSHMRGASGIR
LELIKRMEIFLNAGVTPYVYEFGSIGASGDLVPLSYITGSLIGLDPSFK
VDFNGKEMDAPTALRQLNLSPLTLLPKEGLAMMNGTSVMTGIAANCVYD
TQILTAIAMGVHALDIQALNGTNQSFHPFIHNSKPHPGQLWAADQMISL
LANSQLVRDELDGKHDYRDHELIQDRYSLRCLPQYLGPIVDGISQIAKQ
IEIEINSVTDNPLIDVDNQASYHGGNFLGQYVGMGMDHLRYYIGLLAKH
LDVQIALLASPEFSNGLPPSLLGNRERKVNMGLKGLQICGNSIMPLLTF
YGNSIADRFPTHAEQFNQNINSQGYTSATLARRSVDIFQNYVAIALMFG
VQAVDLRTYKKTGHYDARACLSPATERLYSAVRHVVGQKPTSDRPYIWN
DNEQGLDEHIARISADIAAGGVIVQAVQDILPCLHLPETG

### RBC Pretreatment

Red blood cells were separated from peripheral blood of C57BL/6J mice by density gradient centrifugation. The separated red blood cells were washed with PBS for 3 times. Then the RBCs were pretreated with 1 mM TCEP for 1 hr at RT. Then the pretreated RBCs were washed with PBS for 3 times. GAASK-mal linker (Synthesized by Beijing Scilight Biotechnology Led. Co.) (see FIG. 2) was synthesized with more than 99% purity. GAASK-mal was dissolved in 37° C. phosphate buffer to a final concentration of 100 μM. Then 1×10⁹ RBCs were contacted with 50 μM GAASK-mal for 30 mins at 37° C. Then the obtained RBCs were washed with PBS for 3 times. The RBCs were used immediately or stored at 4° C. for further use.

### GAASK-Mal-Mediated Labeling of RBCs

Reactions were performed in a total volume of 200 μL at 4° C. for 2 hrs in PBS buffer while being rotated at a speed of 10 rpm. The concentration of GAASK-mal-RBCs in the reaction was 1×10⁹/mL. In the reaction system, the final concentration of mg SrtA was 10 μM and the PAL-LEPTG substrate was 10 mM, respectively. After the reaction, the labeling efficiency of RBCs was analyzed by FACS using Beckman Coulter CytoFLEX LX.

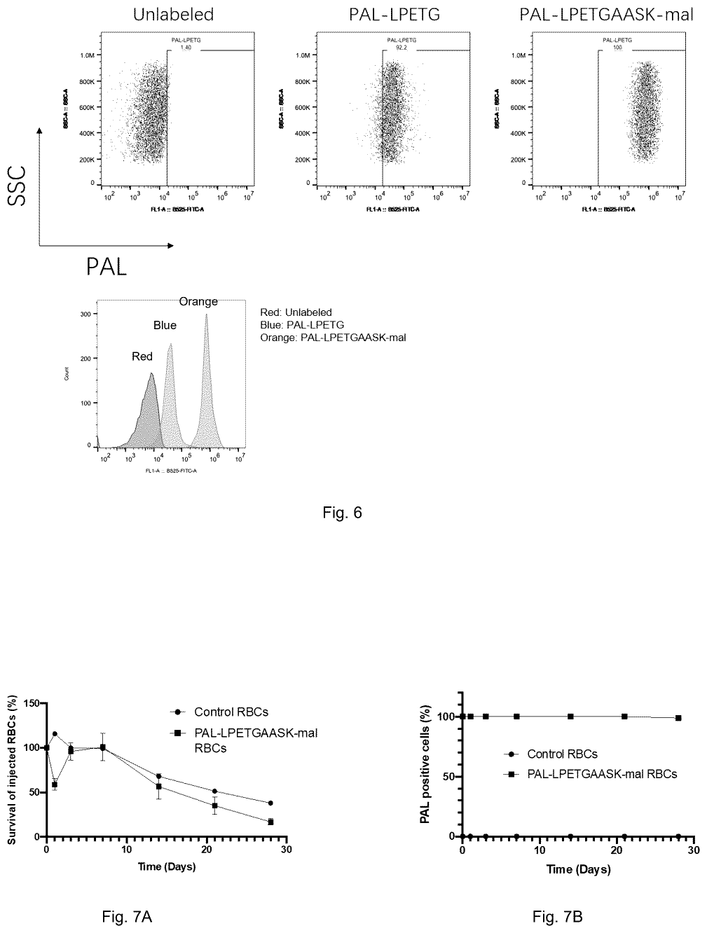

Then labeling efficacy of PAL on the RBC membrane was characterized. The results showed that about 100% of natural RBCs were PAL-LPETGAASK-mal-labeled in vitro, and the labeling efficacy of PAL-LPETGAASK-mal-labelling was about 25 times higher than the efficacy of the PAL-LEPTG (see FIG. 6).

The Life-Span of RBCs Labeled with PAL-LPETGAASK-Mal In Vivo

To assess the life-span of the above obtained surface modified RBCs in vivo, PAL-LPETGAASK-mal tagged mouse RBCs (Dosage: 1×10⁹/mouse) were labeled by a fluorescent dye cell trace Far Red and transfused into recipient mice. The percentage of Far Red and PAL-LPETGAASK-mal positive RBCs in vivo was analyzed periodically through FACS. PAL-LPETGAASK-mal labeled RBCs not only showed the same lifespan as that of the control groups (mice transfused with RBCs without a PAL-LPETGAASK-mal label) but also exhibited sustained signals in circulation for 28 days (see FIGS. 7A-7B).

### Example 3. Preparation of RBCs Labeled with HPV16-MHC1 by Using GAASK-Mal

### Purification of HPV16-MHC1 Protein

The amino acid sequence of HPV16-MHC1-LEPTG is as shown in SEQ ID NO: 11 below:

MSRSVALAVLALLSLSGLEAYMLDLQPETGCGASGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFFTSVSRPGRGEPRFIAVGYVDDTQF
VRFDSDAASQRMEPRAPWIEQEGPEYWDGETRKVKAHSQTHRVDLGTLR
GCYNQSEAGSHTVQRMYGCDVGSDWRFLRGYHQYAYDGKDYIALKEDLR
SWTAADMAAQTTKHKWEAAHVAEQLRAYLEGTCVEWLRRYLENGKETLQ
RTDAPKTHMTHHAVSDHEATLRCWALSFYPAEITLTWQRDGEDQTTELV
ETRPAGDGTFQKWAAVVVPSGQEQRYTCHVQHEGLPKPLTLRWEMLPET
G

After being separated from cells by centrifugation and microfiltration, the supernatant was loaded onto the IMAC Bestarose FF column (Bestchrom, Shanghai, China) with Ni²⁺ ion equilibrated with binding buffer (20 mM Tris-HCl, 500 mM NaCl, pH7.6). The column was washed by the binding buffer and then eluted by elution buffer 1 (20 mM Tris-HCl, 500 mM NaCl, 30 mM imidazole, pH7.6) until UV absorbance at 280 nm became stable. The protein was collected with elution buffer 2 (20 mM Tris-HCl, 500 mM NaCl, 100 mM imidazole, pH7.6).

The protein fraction was then diluted with ddH₂O (1:1), and the loaded onto Diamond Mix-A column (Bestchrom, Shanghai, China) equilibrated with binding buffer (10 mM Tris-HCl, 250 mM NaCl, pH7.6). After being washed by the binding buffer and eluted by elution buffer 1 (13.3 mM Tris-HCl, 337.5 mM NaCl, pH7.6), the target protein was eluted with elution buffer 2 (20 mM Tris-HCl, 2000 mM NaCl, pH7.6), and then concentrated with Amicon Ultra-15 Centrifugal Filter Unit (Millipore, Darmstadt, Germany).

Concentrated protein was loaded to Chromdex 200 pg (Bestchrom, Shanghai, China) equilibrated with PBS, and the target protein fractions were collected. The protein was concentrated and stored at −80° C.

### RBC Pretreatment

Red blood cells were separated from peripheral blood of C57BL/6J mice by density gradient centrifugation. The separated red blood cells were washed with PBS for 3 times. Then the RBCs were pretreated with 1 mM TCEP for 1 hr at RT. Then the pretreated RBCs were washed with PBS for 3 times. GAASK-mal linker (Synthesized by Beijing Scilight Biotechnology Led. Co.) (see FIG. 2) was synthesized with more than 99% purity. GAASK-mal was dissolved in 37° C. phosphate buffer to a final concentration of 100 μM. Then 1×10⁹ RBCs were contacted with 50 μM GAASK-mal for 30 mins at 37° C. Then the obtained RBCs were washed with PBS for 3 times. The RBCs were used immediately or stored at 4° C. for further use.

### GAASK-Mal-Mediated Labeling of RBCs

Reactions were performed in a total volume of 200 μL at 4° C. for 2 hrs in PBS buffer while being rotated at a speed of 10 rpm. The concentration of GAASK-mal-RBCs in the reaction was 1×10⁹/mL. In the reaction system, the final concentration of mg SrtA was M and the HPV16-MHC1-LEPTG substrate was in the range of 0.1 μM-100 mM. After the reaction, the labeling efficiency of RBCs was analyzed by FACS using Beckman Coulter CytoFLEX LX.

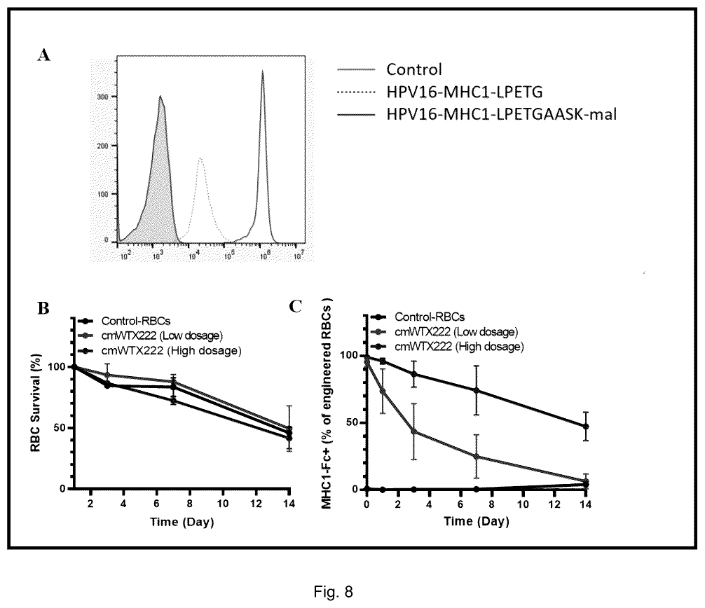

The labeling efficacy of HPV16-MHC1 on the RBC membrane was characterized. The results showed that the labeling efficacy by HPV16-MHC1-LPETGAASK-mal-labelling is about 50 times higher than the efficacy by the HPV16-MHC1-LEPTG (see FIG. 8A). The life-span of RBCs labeled with HPV16-MHC1-LPETGAASK-mal in vivo

To assess the life-span of the above obtained surface modified RBCs in vivo, HPV16-MHC1-LPETGAASK-mal tagged monkey RBCs (High dosage: 7.5×10¹⁰/monkey, low dosage: 1.5×10¹⁰/monkey) were labeled by a fluorescent dye CellTrace Far Red and transfused into recipient mice. The percentage of Far Red positive and HPV16-MHC1-LPETGAASK-mal positive RBCs in vivo was analyzed periodically through FACS. HPV16-MHC1-LPETGAASK-mal labeled RBCs not only showed the same lifespan as that of the control groups (mice transfused with RBCs without a HPV16-MHC1-LPETGAASK-mal label) but also exhibited sustained signals in circulation for 14 days (see FIGS. 8B-8C).

### Example 4. Preparation of Other Mammalian Cells Labeling with eGFP by Using GAAS-mal

To verify whether this method is applicable to other cells, the method was tested on T cells, B cells, monocytes, NK cells, and megakaryocytes.

PBMCs were isolated from healthy human peripheral blood. Whole blood was diluted 1:1 with phosphate buffer, and peripheral blood mononuclear cells (PBMCs) were isolated by centrifugation at 1200×g for 15 minutes using a lymphocyte separation solution (Lymphoprep™, STEMCELL Technologies) and a lymphocyte separation tube.

The purpose of this step was to achieve a density gradient centrifugation of the cell components by the lymphocyte separation solution, and to separate the PBMCs from different cells such as red blood cells and platelets to ensure subsequent enrichment of the T cells.

T cell isolation: the antigen on the cell surface was bound to the corresponding biotin-labeled antibody, and the biotin was bound to the streptavidin-labeled magnetic beads to isolate the lineage of the cell under the action of magnetic force. Cells with specific surface markers were isolated by magnetic beads.

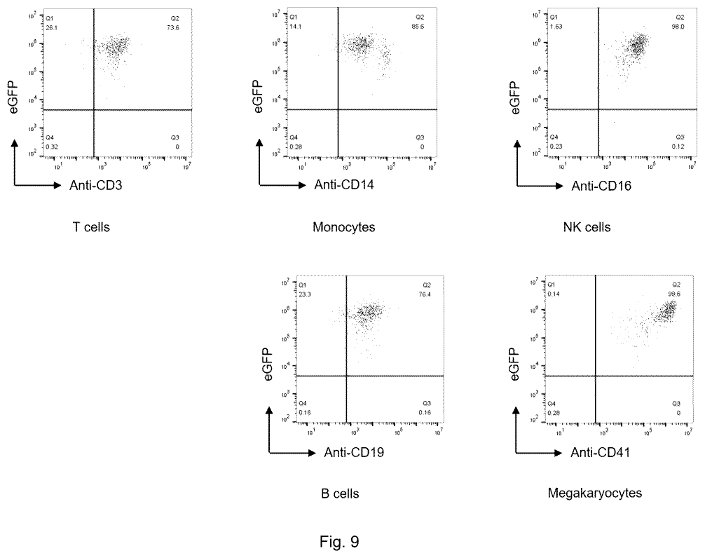

Then, cells labeled with eGFP were prepared as described in Example 1 with T cells, B cells, monocytes, NK cells, or megakaryocytes replacing RBCs. After the reaction, the labeling efficacy was detected by flow cytometry. The results showed that the modification method is also applicable to these cell types and showed good labeling efficiency (see, FIG. 9).

### Example 5. Conjugating Anti PD1 mAb to Red Blood Cells for Treating Tumor

Purification of Anti PD1 mAb-LPETG Protein

An amino acid sequence of Heavy chain-LPETG of anti PD1 mAb-1 is as shown in SEQ ID NO: 12 below:

QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGKGGGGSLPETGG

An amino acid sequence of Light chain of anti PD1 mAb-1 is as shown in SEQ ID NO: 16 below:

EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

A nucleotide sequence encoding Heavy chain-LPETG of anti PD1 mAb-1 is as shown in SEQ ID NO: 13 below

atggattggacatggagagtgttttgtctgcttgcggtgacacctggcg
ctcatcctcaggtacaactggtccagtcaggcgtcgaggttaaaaagcc
cggtgcctcagtgaaggtctcctgcaaagccagtggttacaccttcact
aattactatatgtattgggtcagacaggcacccggccagggtcttgaat
ggatgggaggcatcaatcccagcaatggagggacaaacttcaatgaaaa
gttcaaaaatcgcgtgaccctcacaaccgactcctccacaacaacagca
tatatggaactgaagtctcttcagtttgatgacaccgccgtctactact
gcgcccgacgagactaccggttcgatatgggattcgactattggggtca
ggggacgacagtgacagtctcctccgcatctacaaagggaccatcagtg
tttcctctggccccgtgctcccgctctaccagcgaatctaccgcagcat
tgggctgtttggtgaaggactacttccctgagccagtgaccgtgtcctg
gaacagcggggcactcactagcggagtgcacacatttccagccgtgctt
caaagcagcggtctctattccctctccagcgtggtgactgtgccatcca
gctctctcggcacgaagacctatacctgcaacgtggaccacaagccttc
aaataccaaggtggataaacgcgtcgaatccaaatatggcccaccctgc
ccgccatgtccagctccagagtttctcgggggcctagcgtgttcttgtt
tccccccaaacccaaggacacgctcatgatttccagaacacctgaagtt
acctgcgtcgtcgtagacgtcagtcaggaggacccggaagttcagttta
actggtacgtggacggtgttgaggtgcataatgccaaaaccaagccaag
agaagagcagttcaattctacatatcgagtggtgtcagtcctgacagtt
ctccaccaggactggttgaacggcaaagagtacaaatgcaaggtctcca
acaaaggattgccgagcagtatagagaaaacaattagtaaagctaaggg
tcagcccagagagccccaggtgtacacgttgccaccttcacaggaagag
atgacgaagaaccaagtgtctctgacctgcctggtgaagggcttctacc
catcagacattgcagtggagtgggaaagcaatgggcagcctgagaacaa
ctacaaaactacaccccccgtcctggacagcgatggcagtttcttcctg
tactcaaggttgactgtggacaaaagcagatggcaggaaggcaatgtgt
tcagctgtagtgtgatgcacgaggccctccacaatcattacactcagaa
atccctctctctgagcctgggaaagGGTGGAGGTGGATCTCTACCAGAA
ACAGGTGGA

A nucleotide sequence encoding Light chain of anti PD1 mAb-1 is as shown in SEQ ID NO: 17 below

ATGgGCTGGTCCTGCATCATTCTGTTTCTGGTGGCCACAGCCACCGGCG
TGCACTCTgagattgttctgacccaatctcctgcaacactgagcctgtc
ccccggagagagggcaaccctctcctgccgcgccagtaagggagtgtct
cacgtctggatattcctatctccattggtatcagcagaagcctggccag
gcgcccaggcttctcatctaccttgcttcctatctggagtcggggtgcc
agcccgcttttctgggtccggctctggtaccgacttcaccctgacaatt
tcctcactggagcctgaggatttcgctgtctactattgtcaacattcca
gagaccttcccctgacctttggagggggtacaaaagtggagataaaacg
aacagtggccgccccatctgtattcatctttcctccatctgatgaacag
cttaagtccggcactgcttctgttgtttgcctgcttaacaatttttacc
cgcgggaagccaaggtgcaatggaaagtggacaacgccctgcagtctgg
taacagtcaggaaagcgtgactgaacaggacagcaaagactccacatac
tccttgagttccaccttgacgctcagtaaggcagactacgagaagcaca
aggtttatgcctgtgaagtgactcaccagggcctctctagcccagtgac
taagtccttcaaccgcggggagtgt

An amino acid sequence of Heavy chain-LPETG of anti PD1 mAb-2 is as shown in SEQ ID NO: 14 below:

QMQLVQSGAEVKKPGASVKLSCKASGYIFQSYYIHWVRQAPGQGLEWVG
VINPSGGSTTYAQKFQGRVTMTRDTSISTAYMELSRLRSDDTVVYYCAR
GSYSSGWDYYYYYGMDVWGKGTLVTVSSASTKGPSVFPLAPCSRSTSES
TAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNH
YTQKSLSLSLGKggggslpetgg

An amino acid sequence of Light chain of anti PD1 mAb-2 is as shown in SEQ ID NO: 18 below:

DVVMTQSPLSLPVTPGEPASISCRSSQSLLHSQGYNYLDWYLQKPGQSP
QLLIYLGSNRASGVPDRFSGSGSGTDFTLKISRVEAEDVGVYYCMQALQ
TPWTFGQGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

A nucleotide sequence encoding Heavy chain-LPETG of anti PD1 mAb-2 is as shown in SEQ ID NO: 15 below:

ATGTACCTGGGCCTGAACTGTGTGTTCATCGTGTTTCTGCTGAAGGGCG
TGCAGTCCCAGATGCAGCTGGTGCAGTCCGGCGCCGAGGTGAAGAAGCC
TGGCGCTTCCGTGAAGCTGTCCTGTAAGGCCTCCGGCTACATCTTCCAG
AGCTACTACATCCACTGGGTGAGGCAGGCTCCCGGACAGGGACTGGAAT
GGGTGGGCGTGATTAACCCCTCCGGCGGCTCCACCACCTATGCTCAGAA
GTTCCAGGGCAGGGTGACCATGACCAGGGACACCTCCATCTCCACCGCC
TACATGGAGCTGTCCAGGCTGAGGTCCGACGACACCGTGGTGTACTACT
GCGCTAGGGGCTCCTACTCCTCCGGCTGGGATTACTACTATTACTACGG
CATGGACGTGTGGGGCAAGGGCACCCTGGTGACCGTGAGCTCCGCTAGC
ACAAAGGGACCTTCCGTGTTCCCACTGGCCCCCTGCTCCAGAAGCACAT
CTGAGTCCACCGCCGCTCTGGGCTGTCTGGTGAAGGACTACTTCCCTGA
GCCAGTGACCGTGTCCTGGAACAGCGGCGCCCTGACATCCGGAGTGCAC
ACCTTTCCCGCCGTGCTCCAGTCCAGCGGACTGTACAGCCTGTCTTCCG
TGGTGACAGTGCCCAGCTCTTCCCTGGGCACCAAGACATATACCTGCAA
CGTGGACCATAAGCCTAGCAATACCAAGGTGGATAAGAGGGTGGAGTCT
AAGTACGGACCACCTTGCCCACCATGTCCAGCTCCTGAGTTTCTGGGAG
GACCATCCGTGTTCCTGTTTCCTCCAAAGCCTAAGGACACCCTGATGAT
CTCTCGGACACCTGAGGTGACCTGCGTGGTGGTGGACGTGTCCCAGGAG
GACCCCGAGGTGCAGTTCAACTGGTACGTGGATGGCGTGGAGGTGCACA
ATGCTAAGACCAAGCCAAGAGAGGAGCAGTTTAATAGCACATACCGCGT
GGTGTCTGTGCTGACCGTGCTGCATCAGGATTGGCTGAACGGCAAGGAG
TATAAGTGCAAGGTGAGCAATAAGGGCCTGCCCAGCTCTATCGAGAAGA
CAATCTCTAAGGCTAAGGGACAGCCTCGCGAGCCACAGGTGTACACCCT
GCCCCCTTCCCAGGAGGAGATGACAAAGAACCAGGTGAGCCTGACCTGT
CTGGTGAAGGGCTTCTATCCATCTGACATCGCTGTGGAGTGGGAGTCCA
ACGGCCAGCCCGAGAACAATTACAAGACCACACCACCCGTGCTGGACTC
TGATGGCTCCTTCTTTCTGTATTCCAGGCTGACAGTGGATAAGAGCCGG
TGGCAGGAGGGCAACGTGTTTAGCTGCTCTGTGATGCACGAGGCTCTGC
ACAATCATTATACCCAGAAGTCCCTGAGCCTGTCTCTGGGCAAGGGTGG
AGGTGGATCTctaccagaaacaggtgga

A nucleotide sequence encoding Light chain of anti PD1 mAb-2 is as shown in SEQ ID NO: 19 below

ATGGTGAGTACTCCACAATTTCTAGTGTTTCTCCTGTTCTGGATCCCTG
CCTCCAGAGGCGATGTCGTGATGACCCAGTCCCCTCTGTCCCTGCCTGT
GACACCAGGCGAACCCGCTTCCATCTCCTGCAGATCCTCTCAGAGCCTG
CTGCATTCCCAGGGCTACAACTACCTGGATTGGTATCTGCAGAAGCCTG
GCCAGTCTCCTCAGCTGCTGATCTACCTGGGCTCCAACAGAGCCTCCGG
CGTGCCCGATCGCTTTTCTGGTTCTGGATCTGGCACCGACTTTACACTG
AAGATCAGCCGGGTGGAGGCCGAGGACGTGGGCGTTTACTACTGCATGC
AGGCCCTGCAGACCCCTTGGACCTTCGGCCAGGGAACCAAAGTGGAAAT
CAAGCGGACCGTGGCTGCTCCTTCTGTGTTCATCTTCCCTCCTTCCGAC
GAGCAGCTGAAAAGCGGCACCGCTTCCGTGGTGTGCCTGCTGAACAACT
TCTACCCTAGAGAAGCCAAGGTGCAGTGGAAGGTGGACAATGCTCTGCA
ATCTGGCAACTCCCAAGAGTCCGTGACAGAACAGGACTCCAAGGACTCT
ACCTACTCTCTGTCCTCCACCCTGACCCTGTCTAAGGCCGACTACGAGA
AGCACAAGGTCTACGCCTGCGAGGTGACCCACCAGGGCCTGAGCAGTCC
CGTGACCAAGTCCTTCAACCGGGGCGAGTGT

Nucleotide sequences encoding the above heavy chains or light chains were inserted into the expression vector pcDNA3.1, respectively. Each of successfully constructed vectors was transfected into CHO-S cells using ExpiCHO™ Expression System (ThermoFisher), according to manufacturer's instructions. The transfected cells were cultured in ExpiCHO™ Expression Medium in order to express corresponding heavy chains or light chains and thus assemble corresponding anti PD1 antibodies named as anti PD1 mAb-LPETG due to a label LPETG fused to C-terminal of heavy chain of the antibodies.

Then culture supernatants with anti PD1 mAb-LPETG were harvested and purified by using Protein A affinity chromatography (Cytiva, USA), Q Sepharose FF column (Cytiva, USA), and Bestdex G-25 (Bestchrom, Shanghai, China), according to manufacturer's instructions. The purified target proteins were concentrated and stored at −80° C.

Labeling RBCs with Anti PD1 mAb-LPETG by Using GAASK-Mal

Red blood cells were separated from peripheral blood of C57BL/6J mice, rats and human by density gradient centrifugation, respectively. The separated red blood cells were washed with PBS for 3 times. Then the RBCs were pretreated with 2.5 mM TCEP for 1 hr at RT. Then the pretreated RBCs were washed with PBS for 3 times. Then, the RBCs were modified with GAASK-mal, as disclosed in Example 1, and the obtained modified RBCs named as GAASK-mal-RBCs. Then GAASK-mal-RBCs were used to conjugate anti PD1 mAb-1-LPETG or anti PD1 mAb-2-LPETG via a sortase reaction. The concentration of GAASK-mal-RBCs in the reaction was 1×10⁹/mL. The concentration of mg SrtA was 10 μM and the anti PD1 mAb-1-LPETG substrates or anti PD1 mAb-2-LPETG substrates were in the range of 25 μM-100 μM, respectively. The final obtained RBCs conjugated anti PD1 antibody were named as anti PD1 mAb-RBCs, for example, anti PD1 mAb-1-RBCs and anti PD1 mAb-2-RBCs, stored at 2-8° C.

The amount of anti PD1 mAb-1-LPETG or anti PD1 mAb-2-LPETG conjugated to RBC was measured by sandwiched ELISA. Briefly, coating wells of a PVC microtiter plate with a capture Human PD-1 His tag (ACRO) at 0.5 μg/mL concentration in ELISA coating buffer (pH 9.6, Solarbio) overnight at 4° C.; removing the coating solution and washing the plate twice with 200 μL PBS; blocking the remaining protein-binding sites in the coated wells by adding 200 μL blocking buffer (5% non-fat dry milk/PBS) per well at 37° C. for 1 h; washing the plate twice with 200 μL PBS; lysing anti PD1 mAb-RBCs with RIPA buffer at 4° C. for 10 min. And adding 100 μL the lytic RBCs samples to each well and then incubating for 1 hr at 37° C., in which the test was run in duplicate and comprised positive control and blank control in each plate; removing solutions and washing the plate twice with 200 μL PBS; adding 100 μL of diluted detection anti human FC antibodies (1 μg/mL, eBioscience) to each well and incubating for 1 hr at 37° C.; washing the plate four times with 200 μL PBS; adding TMB solution (SURMODICS) to each well and incubating for 10-15 min, then adding equal volume of stopping solution (Solarbio) and detecting the optical density at 450 nm. Results were disclosed in table 1, from which it can be seen a plenty of anti PD1-mAb were conjugated to RBC from C57BL/6J mice.

TABLE 1
The amount of anti PD1 mAb- LPETG conjugated to RBCs

| | The amount of anti PD1 mAb- LPETG |
|---|---|
| Group | conjugated to RBC (μg/mL) |
| Anti PD1 mAb-1-LPETG | 49~65 |
| Anti PD1 mAb-2-LPETG | 37~129 |

Blockade Efficacy of PD1 mAb-1/2-RBCs

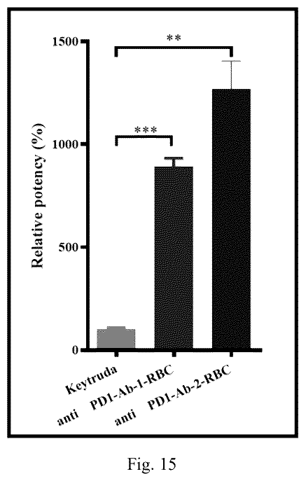

In this test, we evaluating blockade efficacy of anti PD1 mAb-1-RBCs and anti PD1 mAb-2-RBCs by using the PD-1/PD-L1 blockade bioassay kit, according to the manufacturing protocol (genscript, M00613/M00612). Briefly, the assay consists of two genetically engineered cell lines: (1) PD-1 Effector Cells (Jurkat T cells expressing human PD-1 and a luciferase reporter driven by an NFAT response element (NFAT-RE); (2) PD-L1 aAPC/CHO-K1 Cells: CHO-K1 cells expressing human PD-L¹ and an engineered cell surface protein designed to activate cognate TCRs in an antigen-independent manner. Results showed that anti PD1 mAb-1-RBCs and anti PD1 mAb-2-RBCs have enhanced PD-1 blockade efficacy compared to anti PD1 mAb-1 anti PD1 mAb-2, respectively (FIG. 15).

Anti-Tumor Activity of Anti PD1 mAb-2-RBCs In Vivo

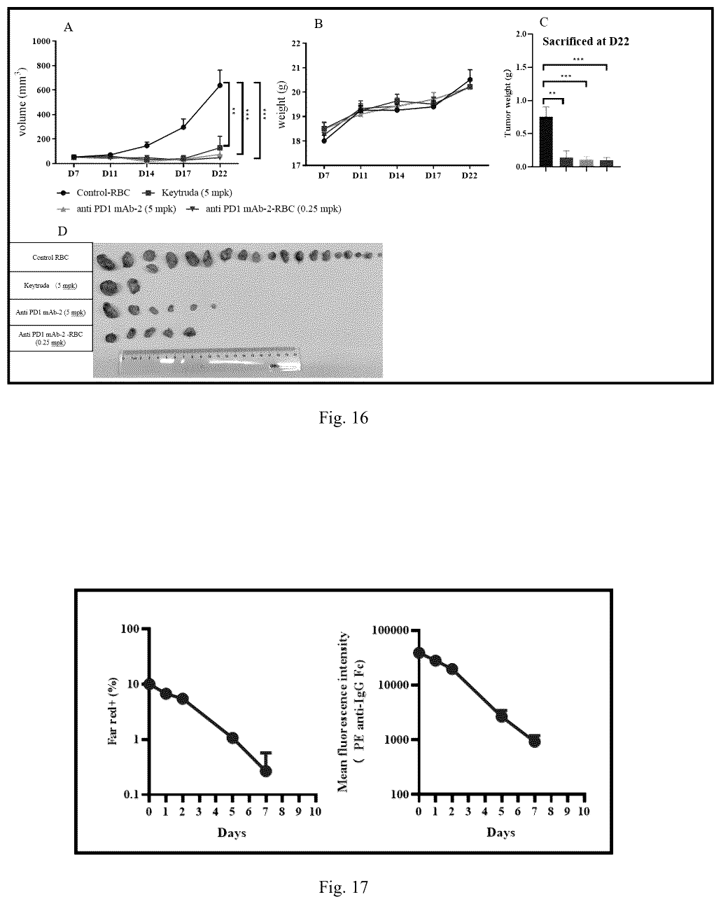

The C57 mice were from Modelorg. According to common techniques, 1×10⁵ MC38 cells in 0.1 mL medium (murine colon adenocarcinoma cell line, from Westlake university) were injected s.c. in the left and right rear flank of C57 mice (6-8 weeks), when the tumors volume (TV) reached about a mean value of 100 mm³, the mice were randomly divided into 4 groups (n=10/group): (1) Control RBCs therapy group; (2) anti PD1-mAb-2-RBC therapy group (0.25 mpk); (3) anti PD1-mAb-2 therapy group (5 mpk); (4) Keytruda (S007467, MSD Ireland) therapy group (5 mpk), and were administered corresponding agents twice a week. Body weight and tumor volume were measured twice a week. The tumor volume was calculated using the empirical formula V=½×[(shortest diameter)²× (the longest diameter)]. After 5 times of administration, the mice were sacrificed and analyzed. Results showed that anti PD1-mAb-2-RBCs therapy groups had enhanced antitumor activity in MC38 tumor models, compared to anti PD1-mAb-2 and Keytruda (FIG. 16), in which, tumor in some mice of groups 2-4 disappeared after administration. The tumor growth inhibition (TGI) of anti PD1-mAb-2-RBCs was 92.6%, while the TGI of anti PD1 mAb-2 was 89.5% and the TGI of Keytruda was 79.9%.

Pharmacokinetics Study of Anti PD1 mAb-RBCs In Vivo

The test evaluated pharmacokinetics study of anti PD1 mAb-RBCs by flow cytometry which measured percentage of Far red (thermo fisher) of anti PD1 mAb-RBCs in C57BL/6J mice. Briefly, C57BL/6J mice were administrated i.v. with 2e9 anti PD1 mAb-1-RBCs. Whole blood was collected into blood collection tubes with K2-EDTA by tail-nick bleed at 0.5 hr (D0), D1, D3 and D7 following the anti PD1 mAb-1-RBCs administration. Samples were analyzed on a Beckman Coulter CytoFLEX LX, and the percentage of Far red positive RBCs and mean fluorescent intensity was analyzed by using FlowJo™ software. Results showed the anti PD1 mAb-1-RBCs could maintain in circulation for at least 7 days (FIG. 17).

### Example 6. Conjugating UOX Proteins to Red Blood Cells for Treating Hyperuricemia and Gout

### Purification of UOX-LPETG Protein

The amino acid sequence of UOX-LPETG is as shown in SEQ ID NO: 20 below:

MSAVKAARYGKDNVRVYKVHKDEKTGVQTVYEMTVCVLLEGEIETSYTK
ADNSVIVATDSIKNTIYITAKQNPVTPPELFGSILGTHFIEKYNHIHAA
HVNIVCHRWTRMDIDGKPHPHSFIRDSEEKRNVQVDVVEGKGIDIKSSL
SGLTVLKSTNSQFWGFLRDEYTTLKETWDRILSTDVDATWQWKNFSGLQ
EVRSHVPKFDATWATAREVTLKTFAEDNSASVQATMYKMAEQILARQQL
IETVEYSLPNKHYFEIDLSWHKGLQNTGKNAEVFAPQSDPNGLIKCTVG
RSSLKSKLAAGSGSGSLPETGG

The nucleotide sequence of UOX-LPETG is as shown in SEQ ID NO: 21 below:

atgtcagcagtaaaggcagcaagatacggtaaagataatgtcagagtct
acaaggttcacaaggacgaaaaaactggtgttcaaacagtttacgaaat
gactgtttgtgttttgttggaaggtgaaatcgaaacttcttacacaaag
gctgataactcagttattgttgcaacagattctattaaaaatactatct
atatcacagctaagcaaaacccagttactccaccagaattgttcggttc
aatcttgggtacacatttcatcgaaaagtacaaccatatccatgctgca
catgttaacatcgtttgtcatagatggactagaatggatattgatggta
aaccacatccacattcttttattagagattcagaagaaaagagaaatgt
tcaagttgatgttgttgagggtaaaggtatcgatatcaagtcttcattg
tcaggtttaactgttttgaagtctacaaattcacaattttggggtttct
tgagagatgaatacactacattgaaggaaacatgggatagaattttatc
tactgatgttgatgctacatggcaatggaagaacttctcaggtttgcaa
gaagttagatctcatgttccaaaatttgatgctacttgggctacagcaa
gagaagttactttgaagacattcgcagaagataactctgcttcagttca
agcaactatgtacaagatggctgaacaaatcttggcaagacaacaattg
atcgaaacagttgaatattcattaccaaataagcattacttcgaaatcg
atttgtcttggcataagggtttgcaaaacactggtaaaaatgctgaagt
tttcgcaccacaatctgatccaaatggtttgattaaatgcacagtcggt
agatcctctttgaagtccaagttagcagcaggttctggttctggttctg
gttctttgccagaaacaggttga

The coding sequence of UOX (Aspergillus flavus uricase) (SEQ ID NO: 21) was codon optimized for expression in E. coli and synthesized by GenScript. Subclones were generated by standard PCR procedure and inserted into the pET-30a vector with C-terminal (GS)3 linker followed by an additional sortase recognition sequence (LPETG). All constructs were verified by sequencing and then transformed in E. coli BL21 (DE3) for protein expression.

A single transformed colony was inoculated into 10 ml Luria-Bertani (LB) medium supplemented with ampicillin (100 μg/ml), grew overnight at 37° C. with 220 rpm shaking. Next day, this 10 ml culture was transferred to 1 L fresh LB medium and grew at 37° C. with 220 rpm shaking until OD600 reached 0.6. The temperature of the culture was then lowered to 20° C. and 1 mM IPTG was added for induction.

After induction, a cell pellet was collected by centrifugation, resuspended in low salt lysis buffer (50 mM Tris 8.8, 50 mM NaCl) and then lysed with sonication. A supernatant containing UOX-LPETG was collected by centrifugation at 10,000 rpm for 1 h, and loaded on a Q Sepharose FF column (Cytiva, Marlborough, USA) pre-equilibrated with a QA buffer (20 mM Tris 8.8). The column was washed with the QA buffer until the absorbance at 280 nm and conductivity became stable and then eluted with a linear gradient of 0-1 M NaCl in 20 mM Tris 8.8. Fractions corresponding to the elution peak were analyzed by SDS-PAGE and the purest fractions were pooled. The pooled elution was diluted by a buffer (20 mM Tris8.0), then loaded on a Diamond MixA colum (Bestchrom) and eluted with a linear gradient of 0-1 M NaCl in 20 mM Tris 8.0. Fractions corresponding to the elution peak were analyzed by SDS-PAGE and the purest fractions were pooled. After adding equal volume buffer (40 mM Tris pH7.5, 2M (NH₄)₂SO₄), the elution sample was loaded on a UniHR Phenyl-80 L column (NanMicr), and washed with 60% gradient buffer B (20 mM Tris 7.5), then eluted with 100% buffer B (20 mM Tris7.5). Concentration of the elution was detected with Amicon Ultra-15 Centrifugal Filter Unit (Millipore, Darmstadt, Germany). Concentrated elution was loaded on a EzLoad 16/60 Chromdex 200 pg (Bestchrom, Shanghai, China) pre-equilibrated with PBS, and then the target protein peak was collected.

Preparation of RBCs Labeling with UOX-LPETG by Using GAASK-Mal Linker

Red blood cells were separated from peripheral blood of C57BL/6J mice, rats and human by density gradient centrifugation, respectively. The separated red blood cells were washed with PBS for 3 times. Then the RBCs were pretreated with 2.5 mM TCEP for 1 hr at RT. The pretreated RBCs were washed with PBS for 3 times, modified with GAASK-mal linker as disclosed in Example 1, finally RBCs modified with GAASK-mal were obtained, name as GAASK-mal-RBCs. Then, 1×10⁹/mL GAASK-mal-RBCs were conjugated with UOX-LPETG via a sortase reaction. In the conjugation reaction, concentration of mg SrtA was 10 μM and the UOX-LPETG substrate was in the range of 25 μM-100 μM. After conjugation, the final products, UOX-RBC, were stored at 2-8° C.

The amount of UOX-LPETG conjugated to RBC was measured by sandwiched ELISA. Specifically, coating wells of a PVC microtiter plate with an anti-UOX antibody-1 (HuaBio) at 0.5 μg/mL concentration in an ELISA coating buffer (pH 9.6, Solarbio) overnight at 4° C.; removing the coating solution, and washing wells of the plate twice with 200 μL PBS; blocking free protein-binding sites in the coated wells with 200 μL blocking buffer (5% non-fat dry milk/PBS) per well at 37° C. for 1 hr; washing the plate twice with 200 μL PBS. UOX-RBCs were lysed by using RIPA buffer (R&D) at 4° C. for 10 min and 100 μL lysed solution was added to each wells of the plate. Each plate comprising positive control (duplicates) and blank control was incubated for 1 hr at 37° C. Solutions were removed and the plate was washed twice with 200 μL PBS. 100 μL diluted detection anti UOX antibody-2 solution (HuaBio, 1 μg/mL, HRP-conjugated) was added to each well and incubated for 1 hr at 37° C. The plate was then washed four times with 200 μL PBS. TMB solution (Solarbio) was added into each well, incubated for 10-15 min, then equal volume of stopping solution (Solarbio) was added, and the optical density at 450 nm was detected. As shown in table 2, a plenty of UOX proteins were conjugated to RBC derived from C57BL/6J mice, rats and human (table 2).

TABLE 2
The amount of UOX proteins conjugated to RBC

| | The amount of UOX proteins conjugated to RBC |
|---|---|
| Group | (μg/10¹⁰ RBC) |
| Mouse UOX-RBC | 30.0~150.0 |
| Rat UOX-RBC | 10.6~137.2 |
| Human UOX-RBC | 10.1~108.2 |

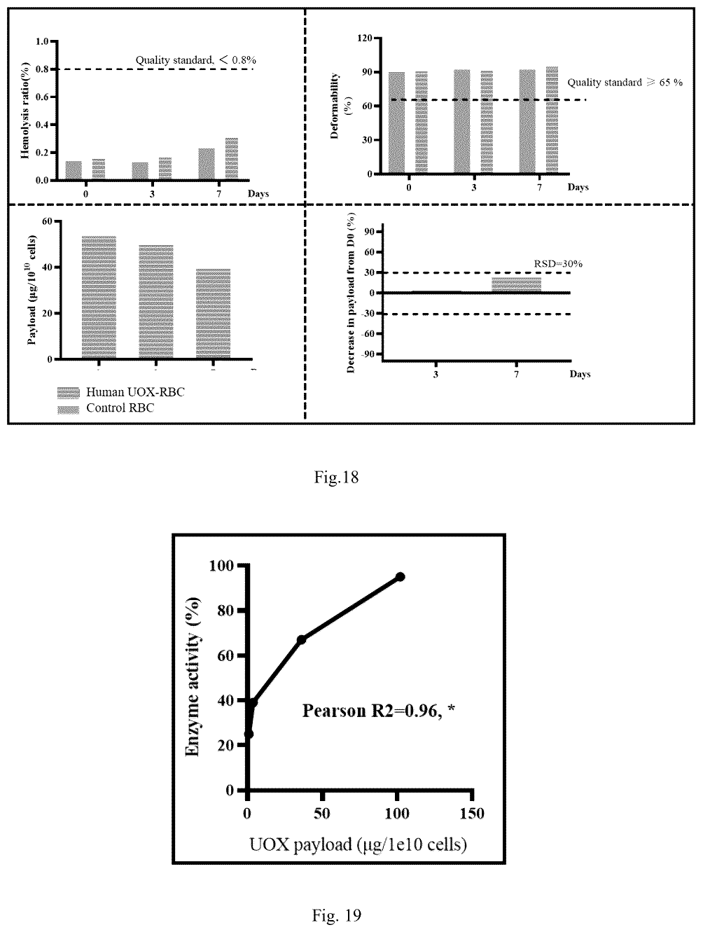

The inventors further characterized UOX-RBCs and detected hemolysis rate (RUI ER DA) and deformability (Changchun Huili) of the UOX-RBCs during the storage according to the manufacturer's instructions. Results showed in FIG. 18, in which the top panels showed that there were no any significant changes between UOX-RBCs and RBCs in terms of hemolysis rate and deformability, and the bottom panels showed UOX proteins covalently conjugated to RBCs did not affect stability of RBCs, the UOX-RBCs were stable during storage until D7 in vitro.

### Enzymatic Activity of UOX-RBCs In Vitro

UOX can catalyze oxidation of uric acid into allantoin, decrease concentration of uric acid in blood and therefore be used for treat hyperuricemia. In this test, enzymatic activity of UOX conjugated on RBCs was detected in vitro. Briefly, cultured 1×5e7 UOX-RBCs were incubated with 30 mg/L uric acid in culture media at 37° C. for 30 min. The uric acid concentration was detected after incubation to determine the conversion rates of UA by the UOX conjugated on RBCs in vitro, according to commercial assay (abcam, ab65344). UOX proteins were used as positive control to calculate the relative enzyme activity of UOX conjugated on RBCs. Results showed that the enzyme activity of UOX conjugated on human RBCs was not impacted by RBCs and was proportional to the UOX payload on RBCs (FIG. 19).

### Therapeutic Efficiency of UOX-RBCs In Vivo

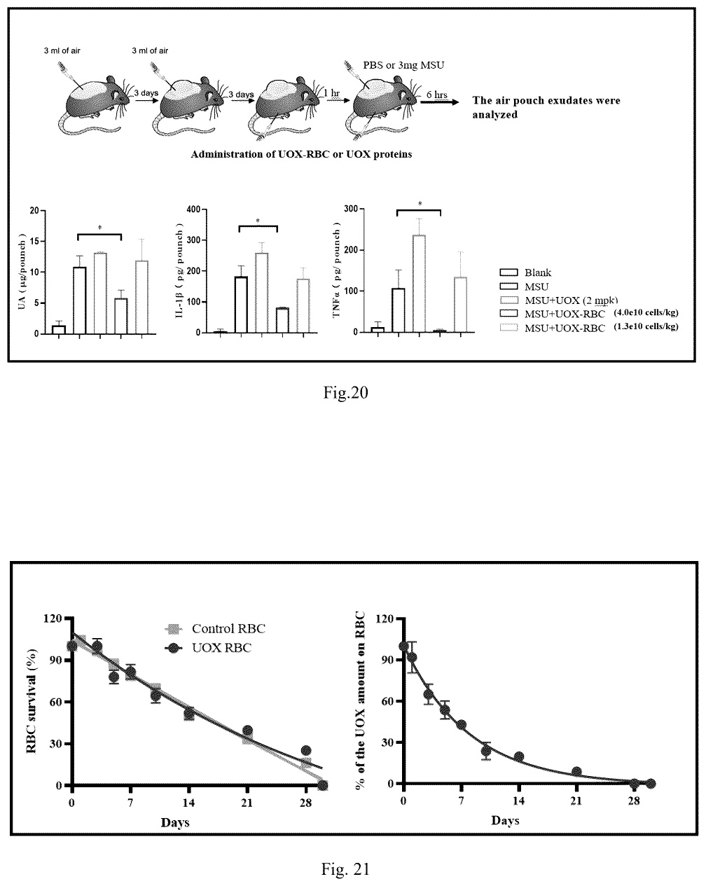

We also assessed therapeutic efficiency of UOX-RBCs in a mouse model of gout. The monosodium urate (MSU) air pouch is a well-established model for studying gout, induced by subcutaneous injection of air into mice. Experimental scheme please refer to FIG. 20. In which, 15 mice were divided into 5 groups. 3 mL filtered air was injected subcutaneously into mice to create a pseudosynovial cavity on day 0. A second air injection (3 ml) was given on day 3 to keep the pouch inflated. On day 6, the animals were administrated with UOX-RBCs (high dosage, 4.0e10/kg; low dosage, 1.3e10/kg). 1 h later, the mice were received an i.p. injection of MSU (3 mg) suspended in PBS, and blank group is only received PBS. Another 6 h later, the pouch exudate was collected after injecting 1 ml PBS for measurement of cytokines levels (IL-1β, TNFα) and the infiltrating leukocytes. Results showed that unlike the UOX proteins itself, UOX-RBCs can remove the uric acid crystals in the joints due to the extensive circulatory range and reduce local inflammation in the air pouch (FIG. 20). Therapeutic efficiency of UOX-RBCs was also dose-dependent.

### Pharmacokinetics Study of UOX-RBCs In Vivo

The test evaluated pharmacokinetics study of UOX-RBCs by detecting percentage mouse UOX-RBCs in vivo through flow cytometry. Briefly, 10 NSG mice were divided into 2 groups. The animals were administrated i.v. with 1e9 mouse UOX-RBC, equivalent control RBCs were used as control. Transfused RBCs were labeled by a fluorescent dye CellTrace Far Red (thermofisher). Whole blood was collected into blood collection tubes with K2-EDTA by tail-nick bleed at 0.5 hr (D0), D1, D3, D7, D14 and D21 following the UOX-RBCs administration. Samples were analyzed on a Beckman Coulter CytoFLEX LX, and the percentage and mean fluorescent intensity of UOX-RBC was analyzed by using FlowJo™ software. Results showed that the UOX-RBCs had the same survival rate as control RBCs and prolonged the life span compared to UOX proteins alone. (FIG. 21).

### REFERENCES

[1] J. W. Yoo, D. J. Irvine, D. E. Discher, and S. Mitragotri, “Bio-inspired, bioengineered and biomimetic drug delivery carriers,” Nat. Rev. Drug Discov., vol. 10, no. 7, pp. 521-535, 2011.
[2] J. M. Antos, J. Ingram, T. Fang, N. Pishesha, M. C. Truttmann, and H. L. Ploegh, “Site-Specific Protein Labeling via Sortase-Mediated transpeptidation,” 2017.
[3] J. Shi, L. Kundrat, N. Pishesha, A. Bilate, C. Theile, and T. Maruyama, “Engineered red blood cells as carriers for systemic delivery of a wide array of functional probes,” pp. 1-6, 2014.
[4] Kuba K, Imai Y, Rao S, Gao H, Guo F, et al. 2005. Nat Med 11: 875-9.
[5] Glowacka I, Bertram S, Herzog P, et al. 2010. Journal of Virology 84: 1198-205.
[6] Huang F, Guo J, Zou Z, Liu J, Cao B, et al. 2014. Nat Commun 5: 3595.
[7] Imai Y, Kuba K, Rao S, Huan Y, Guo F, et al. 2005. Nature 436: 112-6.

1. A cell having an agent linked thereto, wherein the agent is linked to at least one membrane protein of the cell via a sortase recognition motif, and the cell comprises a structure of A¹-L¹-GlyₘXₙ-L²-P, in which A¹ represents the agent, L¹ represents the residual part of a sortase recognition motif after a sortase-mediated reaction, Glyₘ represents m glycines with m being 1-5, Xₙ represents n spacing amino acids with n being 0-10, L² is absent or represents the residual part of a first bifunctional crosslinker after crosslinking, and P represents the at least one membrane protein of the cell, wherein Xₙ comprises at least one amino acid having a side chain amino group, and preferably the C-terminal amino acid of Xₙ is an amino acid having a side chain amino group: wherein the first bifunctional crosslinker is an maleimido carbonic acid (C₂₋₈), and crosslinks said side chain amino group and at least one exposed sulfhydryl of the at least one membrane protein;
wherein the sortase recognition motif comprises, or consists essentially of, or consists of an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X is any amino acid, or wherein the sortase recognition motif comprises, or consists essentially of, or consists of an amino acid sequence selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein * represents the optionally substituted hydroxyl carboxylic acid, and X and Y independently represent any amino acid;
wherein L¹ is selected from the group consisting of LPXT, LPXA, LPXS, LPXL, LPXV, LGXT, LAXT, LSXT, NPXT, MPXT, IPXT, SPXT, VPXT, and YPXR, X being any amino acid;
wherein the sortase is a Sortase A (SrtA); and
wherein the agent A¹ is linked to L¹ via a second bifunctional crosslinker being maleimido carbonic acid (C₂₋₈), and the agent A¹ comprises an exposed sulfydryl, wherein the agent comprises a binding agent, a therapeutic agent, or a detection agent.

2.-4. (canceled)

5. The cell of claim 1, wherein the amino acid having a side chain amino group is lysine; wherein the maleimido carbonic acid (C₂₋₈) is 6-Maleimidohexanoic acid or 4-Maleimidobutyric acid; wherein the sortase recognition motif is LPETG, LPET*G with * being 2-hydroxyacetic acid; wherein the L¹ is LPET; wherein the SrtA is a Staphylococcus aureus transpeptidase A, e.g., Staphylococcus aureus transpeptidase A variant (mgSrtA); and wherein the exposed sulfydryl is an exposed cysteine.

6. (canceled)

7. The cell of claim 5, wherein the maleimido carbonic acid (C₂₋₈) is a 6-Maleimidohexanoic acid, wherein the exposed cysteine is a terminal cysteine, preferably a C-terminal cysteine.

8. The cell of claim 1, wherein the binding agent, the a therapeutic agent, or the detection agent, is a protein, a peptide such as an extracellular domain of oligomeric ACE2, an antibody such as anti-PD1 antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme) such as Aspergillus flavus uricase, a hormone, a cytokine, a growth factor, an antimicrobial agent, a probe, a ligand, a receptor, an immunotolerance-inducing peptide, a targeting moiety, a prodrug or any combination thereof.

9. The cell of any of claim 1, wherein the cell comprises a structure of A¹-LPET-GlyₘXₙ-L²-P, wherein the A¹ is selected from PAL (phenylalanine ammonia-lyase), HPV (such as HPV16-MHC1), UOX, or PD1 mAb, and wherein the GlyₘXₙ-L² is GAASK-mal.

10. (canceled)

11. The cell of claim 1, wherein the cell is selected from the group consisting of red blood cells, T cells, B cells, monocytes, NK cells, and megakaryocytes.

12. The cell of claim 11, wherein the cell is red blood cells with a structure selected from PAL-LPET-GAASK-mal-P, HPV-LPET-GAASK-mal-P, HPV16-MHC1-LPET-GAASK-mal-P, UOX-LPET-GAASK-mal-P, or PD1 mAb-1-LPET-GAASK-mal-P.

13. A method for modifying a cell, comprising:
(i) providing GlyₘXₙ-L^{2′}, in which Glyₘ represents m glycines with m being 1-5, and Xₙ represents n spacing amino acids with n being 0-10, and L^{2′} represents the residual part of a first bifunctional crosslinker after linking to GlyₘXₙ, wherein Xₙ comprises at least one amino acid having a side chain amino group, and preferably the C-terminal amino acid of Xₙ is an amino acid having a side chain amino group;
(ii) treating the cell with GlyₘXₙ-L^{2′} under conditions sufficient to link the GlyₘXₙ-L^{2′} to at least one membrane protein of the cell; and
(iii) contacting the treated cell with a sortase substrate that comprises a sortase recognition motif and an agent, in the presence of a sortase under conditions suitable for the sortase to conjugate the sortase substrate to the Glyₘ by a sortase-mediated reaction,
thereby a modified cell with a structure of A¹-L¹-GlyₘXₙ-L^{2′}-P is obtained, wherein A¹ represents the agent, L¹ represents the residual part of a sortase recognition motif after a sortase-mediated reaction, and P represents the at least one membrane protein of the cell,
wherein the sortase is a Sortase A (SrtA),
wherein the sortase recognition motif comprises, or consists essentially of, or consists of an amino acid sequence selected from the group consisting of LPXTG, LPXAG, LPXSG, LPXLG, LPXVG, LGXTG, LAXTG, LSXTG, NPXTG, MPXTG, IPXTG, SPXTG, VPXTG, YPXRG, LPXTS, and LPXTA, wherein X is any amino acid, or wherein the sortase recognition motif comprises, or consists essentially of, or consists of an amino acid sequence selected from the group consisting of LPXT*G, LPXA*G, LPXS*G, LPXL*G, LPXV*G, LGXT*G, LAXT*G, LSXT*G, NPXT*G, MPXT*G, IPXT*G, SPXT*G, VPXT*G, YPXR*G, LPXT*S, and LPXT*A, wherein * represents the optionally substituted hydroxyl carboxylic acid, and X and Y independently represent any amino acid,
wherein L¹ is selected from the group consisting of LPXT, LPXA, LPXS, LPXL, LPXV, LGXT, LAXT, LSXT, NPXT, MPXT, IPXT, SPXT, VPXT, and YPXR, X being any amino acid,
wherein the agent A¹ is linked to L¹ via a second bifunctional crosslinker being a maleimido carbonic acid (C₂₋₈), and the agent A¹ comprises an exposed sulfydryl, wherein the agent comprises a binding agent, a therapeutic agent, or a detection agent.

14. The method of claim 13, wherein before the treating step, the method further comprises a step of pretreating the cell with a reducing agent to form an exposed sulfhydryl.

15. (canceled)

16. The method of claim 13, wherein in step (i), a first bifunctional crosslinker is used to crosslinks said side chain amino group and at least one exposed sulfhydryl of the at least one membrane protein, wherein the first bifunctional crosslinker is maleimido carbonic acid (C₂₋₈), e.g., 6-Maleimidohexanoic acid, 4-Maleimidobutyric acid;
wherein the L¹ is LPET;
wherein the side chain amino group is lysine; and
wherein the SrtA is a Staphylococcus aureus transpeptidase A, e.g., Staphylococcus aureus transpeptidase A variant (mgSrtA).

17.-19. (canceled)

20. The method of claim 13, wherein the maleimido carbonic acid (C₂₋₈) is 6-Maleimidohexanoic acid or 4-Maleimidobutyric acid, and the exposed sulfydryl is an exposed cysteine, more preferably a terminal cysteine, most preferably a C-terminal cysteine.

21. The method of claim 13, wherein the binding agent, the therapeutic agent, or the detection agent, is a protein, a peptide such as an extracellular domain of oligomeric ACE2, an antibody such as anti-PD1 antibody or its functional antibody fragment, an antigen or epitope such a tumor antigen, a MHC-peptide complex, a drug such as a small molecule drug (e.g., an antitumor agent such as a chemotherapeutic agent), an enzyme (e.g., a functional metabolic or therapeutic enzyme) such as Aspergillus flavus uricase, a hormone, a cytokine, a growth factor, an antimicrobial agent, a probe, a ligand, a receptor, an immunotolerance-inducing peptide, a targeting moiety, a prodrug, or any combination thereof.

22. The method of claim 13, wherein the modified cell comprises a structure of A¹-LPET-GlyₘXₙ-L^{2′}-P, wherein the A¹ is selected from PAL (phenylalanine ammonia-lyase), HPV (such as HPV16-MHC1), UOX, or PD1 mAb, and wherein the GlyₘXₙ-L^{2′} is GAASK-mal.

23. (canceled)

24. The method of claim 13, wherein the modified cell is selected from the group consisting of red blood cells, T cells, B cells, monocytes, NK cells, and megakaryocytes.

25. The method of claim 24, wherein the modified cell is red blood cells with a structure selected from PAL-LPET-GAASK-mal-P, HPV-LPET-GAASK-mal-P, HPV16-MHC1-LPET-GAASK-mal-P, UOX-LPET-GAASK-mal-P, or PD1 mAb-1-LPET-GAASK-mal-P.

26. (canceled)

27. A composition comprising the cell of claim 1 and optionally a physiologically acceptable carrier.

28. A method for diagnosing, treating, or preventing a disorder, condition, or disease in a subject in need thereof, comprising administering the cell of claim 1.

29. The method of claim 28, wherein the disorder, condition or disease is selected from the group consisting of tumors or cancers such as cervical cancer, metabolic diseases such as lysosomal storage disorders (LSDs), bacterial infections, virus infections such as coronavirus infection for example SARS-COV or SARS-COV-2 infection, autoimmune diseases, and inflammatory diseases.

30. A method of delivering an agent to a subject in need thereof, comprising administering the cell of claim 1 to the subject.

31. A method of increasing the circulation time or plasma half-life of an agent in a subject, comprising attaching the agent to a cell according to the method of claim 13.

32.-34. (canceled)


SEQUENCE LISTING (21 sequences)

SEQ ID NO: 1 - protein, 206 aa, Staphylococcus aureus
MKKWTNRLMTIAGVVLILVAAYLFSKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 2 - DNA, 618 bp, Staphylococcus aureus
  source 1..618: mol_type = other DNA
atgaaaaaatggacaaatcgattaatgacaatcgctggtgtggtacttatcctagtggcagcatatttgtttgctaaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagcctacagatgtaggagttctagatgaacaaaaaggtaaagataaacaattaacattaattacttgtgatgattacaatgaaaagacaggcgtttgggaaaaacgtaaaatctttgtagctacagaagtcaaa

SEQ ID NO: 3 - protein, 181 aa, Staphylococcus aureus
KPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 4 - DNA, 543 bp, Staphylococcus aureus
  source 1..543: mol_type = other DNA
aaaccacatatcgataattatcttcacgataaagataaagatgaaaagattgaacaatatgataaaaatgtaaaagaacaggcgagtaaagataaaaagcagcaagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 5 - protein, 147 aa, synthetic construct
QAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 6 - DNA, 441 bp, synthetic construct
  source 1..441: mol_type = other DNA
caagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 7 - protein, 148 aa, synthetic construct
MQAKPQIPKDKSKVAGYIEIPDADIKEPVYPGPATREQLNRGVSFAEENESLDDQNISIAGHTFIGRPNYQFTNLKAAKKGSMVYFKVGNETRKYKMTSIRNVKPTAVGVLDEQKGKDKQLTLITCDDLNRETGVWETRKILVATEVK

SEQ ID NO: 8 - DNA, 444 bp, synthetic construct
  source 1..444: mol_type = other DNA
atgcaagctaaacctcaaattccgaaagataaatcgaaagtggcaggctatattgaaattccagatgctgatattaaagaaccagtatatccaggaccagcaacacgtgaacaattaaatagaggtgtaagctttgcagaagaaaatgaatcactagatgatcaaaatatttcaattgcaggacacactttcattggccgtccgaactatcaatttacaaatcttaaagcagccaaaaaaggtagtatggtgtactttaaagttggtaatgaaacacgtaagtataaaatgacaagtataagaaatgttaagcctacagctgtaggagttctagatgaacaaaaaggtaaagataaacaattaacattaattacttgtgatgatcttaatcgggagacaggcgtttgggaaacacgtaaaatcttggtagctacagaagtcaaa

SEQ ID NO: 9 - protein, 253 aa, synthetic construct
MHHHHHHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKAALPETG

SEQ ID NO: 10 - protein, 628 aa, synthetic construct
MHHHHHHSSGLVPRGSGMKETAAAKFERQHMDSPDLGTDDDDKAMADIGSMHHHHHHKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLHLPETG

SEQ ID NO: 11 - protein, 442 aa, synthetic construct
MSRSVALAVLALLSLSGLEAYMLDLQPETGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTTELVETRPAGDGTFQKWAAVVVPSGQEQRYTCHVQHEGLPKPLTLRWEMLPETG

SEQ ID NO: 12 - protein, 458 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSLPETGG

SEQ ID NO: 13 - DNA, 1431 bp, synthetic construct
  source 1..1431: mol_type = other DNA
atggattggacatggagagtgttttgtctgcttgcggtgacacctggcgctcatcctcaggtacaactggtccagtcaggcgtcgaggttaaaaagcccggtgcctcagtgaaggtctcctgcaaagccagtggttacaccttcactaattactatatgtattgggtcagacaggcacccggccagggtcttgaatggatgggaggcatcaatcccagcaatggagggacaaacttcaatgaaaagttcaaaaatcgcgtgaccctcacaaccgactcctccacaacaacagcatatatggaactgaagtctcttcagtttgatgacaccgccgtctactactgcgcccgacgagactaccggttcgatatgggattcgactattggggtcaggggacgacagtgacagtctcctccgcatctacaaagggaccatcagtgtttcctctggccccgtgctcccgctctaccagcgaatctaccgcagcattgggctgtttggtgaaggactacttccctgagccagtgaccgtgtcctggaacagcggggcactcactagcggagtgcacacatttccagccgtgcttcaaagcagcggtctctattccctctccagcgtggtgactgtgccatccagctctctcggcacgaagacctatacctgcaacgtggaccacaagccttcaaataccaaggtggataaacgcgtcgaatccaaatatggcccaccctgcccgccatgtccagctccagagtttctcggcgggcctagcgtgttcttgtttccccccaaacccaaggacacgctcatgatttccagaacacctgaagttacctgcgtcgtcgtagacgtcagtcaggaggacccggaagttcagtttaactggtacgtggacggtgttgaggtgcataatgccaaaaccaagccaagagaagagcagttcaattctacatatcgagtggtgtcagtcctgacagttctccaccaggactggttgaacggcaaagagtacaaatgcaaggtctccaacaaaggattgccgagcagtatagagaaaacaattagtaaagctaagggtcagcccagagagccccaggtgtacacgttgccaccttcacaggaagagatgacgaagaaccaagtgtctctgacctgcctggtgaagggcttctacccatcagacattgcagtggagtgggaaagcaatgggcagcctgagaacaactacaaaactacaccccccgtcctggacagcgatggcagtttcttcctgtactcaaggttgactgtggacaaaagcagatggcaggaaggcaatgtgttcagctgtagtgtgatgcacgaggccctccacaatcattacactcagaaatccctctctctgagcctgggaaagggtggaggtggatctctaccagaaacaggtgga

SEQ ID NO: 14 - protein, 464 aa, synthetic construct
QMQLVQSGAEVKKPGASVKLSCKASGYIFQSYYIHWVRQAPGQGLEWVGVINPSGGSTTYAQKFQGRVTMTRDTSISTAYMELSRLRSDDTVVYYCARGSYSSGWDYYYYYGMDVWGKGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSLPETGG

SEQ ID NO: 15 - DNA, 1449 bp, synthetic construct
  source 1..1449: mol_type = other DNA
atgtacctgggcctgaactgtgtgttcatcgtgtttctgctgaagggcgtgcagtcccagatgcagctggtgcagtccggcgccgaggtgaagaagcctggcgcttccgtgaagctgtcctgtaaggcctccggctacatcttccagagctactacatccactgggtgaggcaggctcccggacagggactggaatgggtgggcgtgattaacccctccggcggctccaccacctatgctcagaagttccagggcagggtgaccatgaccagggacacctccatctccaccgcctacatggagctgtccaggctgaggtccgacgacaccgtggtgtactactgcgctaggggctcctactcctccggctgggattactactattactacggcatggacgtgtggggcaagggcaccctggtgaccgtgagctccgctagcacaaagggaccttccgtgttcccactggccccctgctccagaagcacatctgagtccaccgccgctctgggctgtctggtgaaggactacttccctgagccagtgaccgtgtcctggaacagcggcgccctgacatccggagtgcacacctttcccgccgtgctccagtccagcggactgtacagcctgtcttccgtggtgacagtgcccagctcttccctgggcaccaagacatatacctgcaacgtggaccataagcctagcaataccaaggtggataagagggtggagtctaagtacggaccaccttgcccaccatgtccagctcctgagtttctgggaggaccatccgtgttcctgtttcctccaaagcctaaggacaccctgatgatctctcggacacctgaggtgacctgcgtggtggtggacgtgtcccaggaggaccccgaggtgcagttcaactggtacgtggatggcgtggaggtgcacaatgctaagaccaagccaagagaggagcagtttaatagcacataccgcgtggtgtctgtgctgaccgtgctgcatcaggattggctgaacggcaaggagtataagtgcaaggtgagcaataagggcctgcccagctctatcgagaagacaatctctaaggctaagggacagcctcgcgagccacaggtgtacaccctgcccccttcccaggaggagatgacaaagaaccaggtgagcctgacctgtctggtgaagggcttctatccatctgacatcgctgtggagtgggagtccaacggccagcccgagaacaattacaagaccacaccacccgtgctggactctgatggctccttctttctgtattccaggctgacagtggataagagccggtggcaggagggcaacgtgtttagctgctctgtgatgcacgaggctctgcacaatcattatacccagaagtccctgagcctgtctctgggcaagggtggaggtggatctctaccagaaacaggtgga

SEQ ID NO: 16 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 711 bp, synthetic construct
  source 1..711: mol_type = other DNA
atgggctggtcctgcatcattctgtttctggtggccacagccaccggcgtgcactctgagattgttctgacccaatctcctgcaacactgagcctgtcccccggagagagggcaaccctctcctgccgcgccagtaagggagtgtctacgtctggatattcctatctccattggtatcagcagaagcctggccaggcgcccaggcttctcatctaccttgcttcctatctggagtccggggtgccagcccgcttttctgggtccggctctggtaccgacttcaccctgacaatttcctcactggagcctgaggatttcgctgtctactattgtcaacattccagagaccttcccctgacctttggagggggtacaaaagtggagataaaacgaacagtggccgccccatctgtattcatctttcctccatctgatgaacagcttaagtccggcactgcttctgttgtttgcctgcttaacaatttttacccgcgggaagccaaggtgcaatggaaagtggacaacgccctgcagtctggtaacagtcaggaaagcgtgactgaacaggacagcaaagactccacatactccttgagttccaccttgacgctcagtaaggcagactacgagaagcacaaggtttatgcctgtgaagtgactcaccagggcctctctagcccagtgactaagtccttcaaccgcggggagtgt

SEQ ID NO: 18 - protein, 219 aa, synthetic construct
DVVMTQSPLSLPVTPGEPASISCRSSQSLLHSQGYNYLDWYLQKPGQSPQLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 717 bp, synthetic construct
  source 1..717: mol_type = other DNA
atggtgagtactccacaatttctagtgtttctcctgttctggatccctgcctccagaggcgatgtcgtgatgacccagtcccctctgtccctgcctgtgacaccaggcgaacccgcttccatctcctgcagatcctctcagagcctgctgcattcccagggctacaactacctggattggtatctgcagaagcctggccagtctcctcagctgctgatctacctgggctccaacagagcctccggcgtgcccgatcgcttttctggttctggatctggcaccgactttacactgaagatcagccgggtggaggccgaggacgtgggcgtttactactgcatgcaggccctgcagaccccttggaccttcggccagggaaccaaagtggaaatcaagcggaccgtggctgctccttctgtgttcatcttccctccttccgacgagcagctgaaaagcggcaccgcttccgtggtgtgcctgctgaacaacttctaccctagagaagccaaggtgcagtggaaggtggacaatgctctgcaatctggcaactcccaagagtccgtgacagaacaggactccaaggactctacctactctctgtcctccaccctgaccctgtctaaggccgactacgagaagcacaaggtctacgcctgcgaggtgacccaccagggcctgagcagtcccgtgaccaagtccttcaaccggggcgagtgt

SEQ ID NO: 20 - protein, 316 aa, Aspergillus flavus
MSAVKAARYGKDNVRVYKVHKDEKTGVQTVYEMTVCVLLEGEIETSYTKADNSVIVATDSIKNTIYITAKQNPVTPPELFGSILGTHFIEKYNHIHAAHVNIVCHRWTRMDIDGKPHPHSFIRDSEEKRNVQVDVVEGKGIDIKSSLSGLTVLKSTNSQFWGFLRDEYTTLKETWDRILSTDVDATWQWKNFSGLQEVRSHVPKFDATWATAREVTLKTFAEDNSASVQATMYKMAEQILARQQLIETVEYSLPNKHYFEIDLSWHKGLQNTGKNAEVFAPQSDPNGLIKCTVGRSSLKSKLAAGSGSGSLPETGG

SEQ ID NO: 21 - DNA, 954 bp, Aspergillus flavus
  source 1..954: mol_type = other DNA
atgtcagcagtaaaggcagcaagatacggtaaagataatgtcagagtctacaaggttcacaaggacgaaaaaactggtgttcaaacagtttacgaaatgactgtttgtgttttgttggaaggtgaaatcgaaacttcttacacaaaggctgataactcagttattgttgcaacagattctattaaaaatactatctatatcacagctaagcaaaacccagttactccaccagaattgttcggttcaatcttgggtacacatttcatcgaaaagtacaaccatatccatgctgcacatgttaacatcgtttgtcatagatggactagaatggatattgatggtaaaccacatccacattcttttattagagattcagaagaaaagagaaatgttcaagttgatgttgttgagggtaaaggtatcgatatcaagtcttcattgtcaggtttaactgttttgaagtctacaaattcacaattttggggtttcttgagagatgaatacactacattgaaggaaacatgggatagaattttatctactgatgttgatgctacatggcaatggaagaacttctcaggtttgcaagaagttagatctcatgttccaaaatttgatgctacttgggctacagcaagagaagttactttgaagacattcgcagaagataactctgcttcagttcaagcaactatgtacaagatggctgaacaaatcttggcaagacaacaattgatcgaaacagttgaatattcattaccaaataagcattacttcgaaatcgatttgtcttggcataagggtttgcaaaacactggtaaaaatgctgaagttttcgcaccacaatctgatccaaatggtttgattaaatgcacagtcggtagatcctctttgaagtccaagttagcagcaggttctggttctggttctggttctttgccagaaacaggttga